(19) United States
(12) Patent Application Publication
(10) Pub. No.: US 2021/0207153 A1
(43) Pub. Date: Jul. 8, 2021

(54) SYSTEMS AND METHODS FOR EDITING A PLANT GENOME

(71) Applicant: PIONEER HI-BRED INTERNATIONAL, INC., Johnston, IA (US)

(72) Inventors: David George Charne, Guelph (CA); Wenpin Chen, Brampton (CA); James Brent Gillespie, Toronto (CA); Siva S. Ammirajo Jetty, Johnston, IA (US); Zhan-Bin Liu, Clive, IA (US); Gina Marie Zastrow-Hayes, Urbandale, IA (US)

(73) Assignee: PIONEER HI-BRED INTERNATIONAL, INC., Johnston, IA (US)

(21) Appl. No.: 17/059,707

(22) PCT Filed: May 30, 2019
(86) PCT No.: PCT/US19/34531

§ 371 Date: Nov. 30, 2020

Related U.S. Application Data
(60) Provisional application No. 62/679,337, filed on Jun. 1, 2018.

(51) Int. Cl. C12N 15/82; A01H 6/20

(52) U.S. Cl. CPC C12N 15/8207; A01H 6/20

(57) ABSTRACT

Systems and methods for editing plant genomes include systems and methods for transforming plants generally, and Brassica plants particularly, with genome editing systems including CRISPR-Cas. Systems and methods for editing plant genomes include systems and methods for transforming Brassica microspores with genome editing systems and culturing the plant microspores that increase genome editing frequency.

### FIELD The field relates to transformation and editing plant genomes.

### BACKGROUND

Plant breeding and crop development relies on producing new varieties with improved agronomic characteristics. Introduction of recombinant DNA into plant genomes by genetic transformation methods are desired to enable transformation of a variety of crop species and tissue types. Targeted editing plant genomes often involve plant transformation methods.

One method for transforming plants involve the use of Agrobacterium. Agrobacterium-based methods generally involve at least one T-DNA border into the genome of the recipient plant. Co-cultivation of plant tissues with Agrobacterium may affect the regeneration of a transformed plant from a transformed cell. Transformation methods that do not involve Agrobacterium such as particle bombardment are also used to transform certain plant cells under chosen conditions. To meet the increasing demands of agriculture in the world today, the pace of development of new varieties of plants must be accelerated. Therefore, there exists a need to improve transformation methodologies that are flexible and scalable to increase transformation efficiency to generate new plants.

### SUMMARY

Methods are provided for producing plants with edited genomes. The methods comprise introducing genome editing systems by microprojectile/microparticle bombardment. The methods find use in agriculture, particularly in the development of improved varieties of plants through the incorporation of desirable agronomic traits. The methods involve introducing a genome editing system by microprojectile bombardment into a microspore that is capable of regenerating into a stably transformed plant and regenerating such a plant from the cell.

A method of editing genome of a Brassica plant cell, the method includes culturing isolated microspore from a Brassica plant to produce an isolated pre-incubated microspore; culturing the isolated pre-incubated microspore under conditions of plasmolysis; introducing, under conditions of plasmolysis through bombardment a microparticle comprising a CRISPR-Cas genome editing system into the isolated pre-incubated microspore; culturing under conditions of plasmolysis the pre-incubated microspore comprising a genetic modification introduced by the genome editing system to generate a transformed microspore; culturing the transformed pre-incubated microspore in or on a liquid selection medium to produce an embryo or a tissue; and regenerating a Brassica plant from the embryo or the tissue, wherein the Brassica plant comprises the genetic modification introduced by the genome editing system. In certain embodiments, the genome editing system comprises CRISPR/Cas endonuclease selected from the group consisting of Cas9, Cpf1, or Csm1. In certain embodiments, the genome editing system comprises a guide RNA, a Cas endonuclease, and combination thereof. In certain embodiments, the gene-editing system comprises a ribonucleoprotein complexed with a guide RNA on a microparticle. In certain embodiments, the genome editing system comprises guide RNA and a donor DNA template. In certain embodiments, the embryo is treated with a chromosome doubling agent.

A method of increasing genome editing frequency of Brassica, the method includes culturing isolated microspore obtained from a Brassica plant to produce an isolated pre-incubated microspore that has been conditioned to be embryogenic; introducing, under conditions of plasmolysis, a microparticle carrier comprising a targeted genome editing system into the isolated pre-incubated microspore through bombardment, wherein the microspore does not burst or otherwise damaged upon receiving the microparticle; generating an embryo from the bombarded microspore under suitable culturing conditions; and generating a Brassica plant from the embryo comprising a genetic modification introduced by the genome editing system. In certain embodiments, the genetic modification is introduction of a heterologous DNA through homologous recombination or homology mediated repair. In certain embodiments, the gene-editing system targets Brassica FAD2, FAD3 or a combination thereof. In certain embodiments, the expression cassette further comprises a gene encoding a selectable marker. In certain embodiments, the selectable marker is glyphosate acetyltransferase.

An embryogenic Brassica microspore cell includes a microparticle, wherein the microparticle delivers a genome editing complex comprising a CRISPR-Cas endonuclease, a guide RNA, and optionally a donor template, wherein the Cas endonuclease and guide RNA form a complex with a target genomic DNA in the microspore cell, wherein the microspore cell is embryogenic and capable of developing into an embryo. In certain embodiments, the genome comprises a targeted edit introduced by the Cas endonuclease—gRNA complex.

In certain embodiments, the microspore is present in an osmolytic culture medium. In certain embodiments, the microspore is obtained from a donor Brassica plant that is Canola. In certain embodiments, the microspore is tolerant to a selectable marker. In certain embodiments, the microspore comprises a mutation in Brassica FAD2, FAD3 or a combination thereof.

An aspect of the disclosure is to provide a method of producing a cell transformed with a genome editing system by particle bombardment, comprising: (a) culturing a pre-incubated microspore-derived explant comprising a cell under a condition of plasmolysis for a period of about half an hour to about 4 hours prior to bombardment; (b) introducing a DNA construct by microprojectile bombardment into an exposed cell on a surface of the pre-incubated microspore-derived explant, wherein the explant is under the condition of plasmolysis; and (c) continuing to culture the bombarded pre-incubated microspore-derived explant under the condition of plasmolysis for a period of about 4 hours to about 20 hours, to produce a transformed cell. A pre-incubated microspore-derived explant is a microspore or any tissue derived from the microspore (for example a microspore-derived embryo or a microspore-derived hypocotyl) that has been cultured for a period of time of between 1 day and 30 days prior to bombardment. The condition of plasmolysis can be selected from the group consisting of (a) culturing the explant on osmotic medium and (b) culturing the explant on wetted filter paper. The explant can be a pre-incubated microspore, a pre-incubated microspore-derived embryo, or a pre-incubated microspore-derived hypocotyl. The method can further comprise the steps of regenerating a transformed plant from the transformed cell, comprising: (a) culturing said microspore-derived explant on a regeneration medium to produce a regenerated embryo or tissue; and (b) regenerating a stably transformed plant from said embryo or tissue

An aspect of the disclosure is to provide a method for producing a plant stably transformed with a genome editing system, comprising: (a) introducing a DNA construct by microprojectile bombardment into a pre-incubated explant, which may be a microspore, or a microspore-derived embryo or portion of a microspore-derived embryo; (b) culturing the pre-incubated explant to produce an embryo or tissue; and (c) regenerating a stably transformed Brassica plant from the embryo or tissue. In step (a), a pre-incubated microspore can be produced by culturing a microspore in a culture medium for a period of about two to ten days prior to bombardment. The period can be from about four to eight days, or from seven to eight days. The method can further comprise a step of inducing plasmolysis of the pre-incubated microspore prior to, during and after bombardment. For example, plasmolysis can be induced by (a) culturing the pre-incubated microspore on osmotic medium prior to, during and after bombardment, or (b) culturing the pre-incubated microspores on wetted filter paper prior to, during and after bombardment. The osmotic medium can comprise between about 17 and 19% sucrose and between about 0.8 and 1.6% agar (w/v). The pre-incubated microspore can be cultured on osmotic medium for about between half an hour and four hours prior to bombardment and for about between four hours and twenty hours after bombardment.

In certain embodiments, the method can further comprise a selection step after bombardment comprising culturing the bombarded pre-incubated microspore on a medium comprising a selection agent against a gene encoded by the DNA construct. For example, the selection agent can be selected from the group consisting of kanamycin, G418 and glyphosate. The concentration of G418 can be between about 5 and 10 mg/l. The concentration of glyphosate in the medium can be between about 0.1 mM and 0.2 mM. The method may further comprise a step of orientating the pre-incubated microspore during bombardment so that a surface of the microspore is exposed during the bombardment. The method may further comprise a step of collecting the pre-incubated microspore such that it is viable and embryogenic prior to bombardment. The step of collecting the pre-incubated microspore can be a filtration step or a step of Percoll® gradient centrifugation (Percoll® concentration is 35-45%). The filtration step can be done using a sieve having a pore size of about 15 to 48 μm.

In certain embodiments, the microprojectile bombardment can be conducted using bombardment factors comprising about 12.5 ng to 5 μg of said DNA construct, about 15 μg to 100 μg gold particles per shot at the size of 0.4 micron to 0.6 micron, about 2.5 M CaCl2 and a 650 to 900 psi rupture disk. The step of regenerating a stably transformed plant can comprise culturing the bombarded pre-incubated microspore on a first liquid selection medium for a first period of time, a second liquid selection medium for a second period of time, and then transferring the resistant embryo or tissue derived from the pre-incubated microspore to solid medium for a third period of time. The first period of time can be about 7 days and the pre-incubated microspore can be cultured in darkness. The pre-incubated microspore, or tissue or embryo derived from the pre-incubated microspore, can be cultured in the second liquid selection medium for approximately 14 days in dim light of approximately 240 foot candles or 2,583 Lux. The second liquid medium can be liquid NLN-6.5S and further comprise growth regulators. The growth regulators in the second liquid selection medium can comprise 0.5 mg/L NAA and 0.05 mg/l BAP. Further, the first, the second, or both the first and the second liquid selection media can comprise G418 or glyphosate. Further, the solid medium may comprise growth regulators to induce regeneration, and optionally further comprise a selection agent against a gene encoded by the DNA construct. The solid medium can be MMW medium with indoleacetic acid (IAA), thidiazuron (TDZ) and silver nitrate (AgNO3). The solid medium can further comprise a selection agent against a gene encoded by the DNA construct. The method can further comprise use of a chromosome doubling agent to produce a doubled haploid transgenic plant. The doubling agent can be administered within one day after bombardment and can be administered for approximately 7 days.

Where the bombarded explant is a microspore-derived embryo, the method may comprise: (a) culturing a microspore-derived embryo on osmotic medium for a period of about half an hour to about 4 hours prior to bombardment; (b) introducing a DNA construct by microprojectile bombardment into an exposed surface of the microspore-derived embryo on osmotic medium; (c) continuing to culture the bombarded microspore-derived embryo on osmotic medium for a period of about 4 hours to about 20 hours; (d) culturing said bombarded microspore-derived embryo on regeneration media to produce a regenerated embryo or tissue; and (e) regenerating a stably transformed Brassica plant from the regenerated embryo or tissue. The microspore-derived embryo can be between about 11 and 20 days old. The microspore-derived embryo can be between about 11 and 14 days old. The method can further comprise the step of collecting the embryo using a pipette and transferring the embryo onto filter paper prior to step (a). Step (d) can comprise culturing the embryo on liquid medium for a first period of time and then on solid medium for a second period of time. The first period of time can be about 7 to 14 days. The method can further comprise an optional step of excising a hypocotyl from the regenerated embryo and culturing the hypocotyl on regeneration media. Additionally, the method can further comprise a step of selecting for a transformed embryo comprising culturing the embryo on media supplemented with a selection agent against a gene encoded by the DNA construct. The method can comprise use of a chromosome doubling agent to produce a double haploid transgenic plant.

Where the bombarded explant is a portion of a microspore-derived embryo, the method may comprise: (a) culturing a hypocotyl excised from a microspore-derived embryo on osmotic medium for a period of about half an hour to about 4 hours prior to bombardment; (b) introducing a DNA construct by microprojectile bombardment into an exposed surface of the microspore-derived hypocotyl on osmotic medium; (c) continuing to culture the bombarded microspore-derived hypocotyl on osmotic medium for a period of about 4 hours to 20 hours; (d) culturing said microspore-derived hypocotyl on a regeneration medium to produce regenerated embryos or tissues; and (e) regenerating a stably transformed Brassica plant from said embryo or tissue. The microspore-derived hypocotyl can be excised from a microspore-derived embryo of between about 21 and 26 days. The method can further comprise a step of culturing the microspore-derived hypocotyl on a cell division induction medium comprising plant growth regulators for between about 1 to 20 hours prior to bombardment. Step (d) can comprise culturing the embryo on a first solid medium for a first period of time and then on a second solid medium for a second period of time. The first solid medium can comprise plant growth regulators for bud induction. The second solid medium can be free of plant growth regulators or comprises plant growth regulators for shoot formation. The method can further comprise a step of selecting a transformed embryo or tissue comprising culturing the embryo or tissue on media supplemented with a selection agent against a gene encoded by the DNA construct. The method can further comprise use of a chromosome doubling agent to produce a doubled haploid transgenic plant.

Another aspect of the disclosure is to provide a Brassica cell or a stably transformed Brassica plant produced by any one of the methods described above. The plant or cell can be selected from Brassica napus, Brassica rapa, Brassica juncea, Brassica oleracea, Brassica carinata and Brassica nigra. Progeny of the plant and cell are also provided.

### DETAILED DESCRIPTION

Embodiments are drawn to methods for editing the genes of Brassica plants. The methods find use in agriculture in the development of gene-edited crop plants with improved agronomic characteristics. The methods find particular use in introducing desirable traits into a Brassica plant. Such new traits may be, for example, resistance to an herbicide, resistance to pathogens and insects, modified seed oil composition and the like. In certain embodiments, the methods involve introducing a DNA construct into the genome of a cell of a Brassica plant and regenerating a stably transformed plant from the cell.

“Brassica cell” generally refers to a cell from a Brassica plant or a cell that is produced by in vitro culture methods and is descended from a cell from a Brassica plant.

“Somatic embryo” generally refers to an embryo that develops from a somatic cell. The developmental process by which a somatic embryo develops from a cell is known as “somatic embryogenesis.” Such a “somatic embryo” is distinct from a “zygotic embryo” which develops from a zygote.

“Microspore-derived embryo” generally refers to an embryo that develops from a microspore. Because it develops from a germ cell, such a “microspore-derived embryo” is distinct from both somatic and zygotic embryos which develop from somatic cells and zygotes, respectively.

“Microspore-derived hypocotyl” generally refers to a hypocotyl of an embryo that develops from a microspore.

“Adventitious” generally refers to an organ or other structure of a plant that does not originate in the usual location on the plant body. For example, a shoot that originated from a hypocotyl of a microspore-derived embryo is an “adventitious shoot.”

“Canola” generally refers to a Brassica plant or oil from a Brassica plant wherein the oil must contain less than 2% erucic acid and the solid component of the seed must contain less than 30 micromoles of any one or any mixture of 3-butenyl glucosinolate, 4-pentenyl glucosinolate, 2-hydroxy-3 butenyl glucosinolate, and 2-hydroxy-4-pentenyl glucosinolate per gram of air-dry, oil free solid.

“Organogenesis” generally refers to the developmental process wherein a cell or group of cells gives rise to an organ such as, for example, a shoot, a bud or a root.

“Chromosome doubling” generally refers to that each of the chromosomes in a cell is duplicated resulting in a doubling of the number of chromosomes in the cell.

“Ploidy” generally refers to the number of complete sets of chromosomes in the nucleus of a cell. A “haploid” cell has one set of chromosomes, and a “diploid” cell has two sets.

“Effective amount” generally refers to an amount of an agent, compound or plant growth regulator that is capable of causing the desired effect on an organism. It is recognized that an “effective amount” may vary depending on factors, such as, for example, the organism, the target tissue of the organism, the method of administration, temperature, light, relative humidity and the like. Further, it is recognized that an “effective amount” of a particular agent may be determined by administering a range of amounts of the agent to an organism and then determining which amount or amounts cause the desired effect.

“Pre-incubated microspore-derived explant” generally refers to a microspore or any tissue derived from the microspore (for example a microspore-derived embryo or a microspore-derived hypocotyl) that has been cultured for a period of time of between about 1 day and 30 days prior to bombardment. For example, a pre-incubated microspore may be cultured for about two to ten days from the time of isolation of the microspore from a donor plant. For example, a pre-incubated microspore-derived embryo may be cultured for a period of about 11 days to 20 days from the time of isolation of the microspore from a donor plant. For example, a pre-incubated microspore-derived hypocotyl may be cultured for a period of about 21 to 26 days from the time of isolation of the microspore from a donor plant.

“Progeny” generally refers to descendents of a particular cell or plant which comprise at least a portion of the transgene inserted at the locus of the genome of the T0 plant cell. For example, progeny can be seeds developed on a plant and plants derived from such seeds. For example, progeny of a plant include seeds formed on T0, T1, T2 and subsequent generation plants, and plants derived from such seeds. Progeny also includes seeds formed by cross pollination using pollen of a T0, T1, T2, T3, etc. plant. For example, the progeny can be the result of selfing, outcrossing or backcrossing. The progeny can also include asexually propagated plants or cells derived from T0, T1, T2, etc plants or cells that include at least a portion of the transgene inserted at the locus of the genome in the T0 plant cell. For example, plants produced via cuttings, tissue culture, microspore culture, etc. that comprise at least a portion of the original transgene inserted at the locus of the genome of the T0 plant cell are also considered progeny.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “ guide polynucleotide/Cas complex”, “guide polynucleotide/Cas system” and “guided Cas system” “polynucleotide-guided endonuclease”, and “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13). In some aspects, the guide polynucleotide/Cas endonuclease complex is provided as a ribonucleoprotein (RNP), wherein the Cas endonuclease component is provided as a protein and the guide polynucleotide component is provided as a ribonucleotide.

The terms “target site”, “target sequence”, “target site sequence, ”target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave . The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. In some aspects, the Cas endonuclease may not successfully recognize a target DNA sequence if the target DNA sequence is not adjacent to, or near, a PAM sequence. In some aspects, the PAM precedes the target sequence (e.g. Cas12a). In some aspects, the PAM follows the target sequence (e.g. S. pyogenes l Cas9). The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

In some aspects, a “polynucleotide modification template” is provided that comprises at least one nucleotide modification when compared to the nucleotide sequence to be edited. A nucleotide modification can be at least one nucleotide substitution, addition, deletion, or chemical alteration. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In some aspects, a polynucleotide of interest is inserted at a target site and provided as part of a “donor DNA” molecule. As used herein, “donor DNA” is a DNA construct that comprises a polynucleotide of interest to be inserted into the target site. In some aspects, the donor DNA construct may further comprise a first and a second region of homology that flank the polynucleotide of interest. The first and second regions of homology of the donor DNA share homology to a first and a second genomic region, respectively, present in or flanking the target site of the cell or organism genome. The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963). The amount of homology or sequence identity shared by a target and a donor polynucleotide can vary and includes total lengths and/or regions.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In some aspects, methods and compositions are provided for modifying naturally-occurring polynucleotides or integrated transgenic sequences, including regulatory elements, coding sequences, and non-coding sequences. These methods and compositions are also useful in targeting nucleic acids to pre-engineered target recognition sequences in the genome. Modification of polynucleotides may be accomplished, for example, by introducing single- or double-strand breaks into the DNA molecule.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The HDR pathway is another cellular mechanism to repair double-stranded DNA breaks, and includes homologous recombination (HR) and single-strand annealing (SSA) (Lieber. 2010 Annu. Rev. Biochem. 79:181-211).

Methods for Producing Genome-edited Brassica Plants

Methods are provided for producing genome edited Brassica plants. The methods comprise producing edited plants by introducing a genome editing system, for example, DNA of Cas9, gRNA and selectable marker expression cassettes to microspores by microprojectile bombardment.

CRISPR-Cas Methods for Gene Editing

CRISPR/Cas genome editing system was applied in crop plant improvement including Brassica species after the system was developed. The plant explants used for genome editing were somatic cells on all published papers and patents. Compared to somatic cells with 2-copy of genomes, male gamete cells (microspores) have one copy of genome. Genome editing efficiency would be higher in microspores than that in somatic cells. Edited plants would be homozygous on edited genome sites by doubling chromosome number after treating edited cells or plants.

Examples of a Cas endonuclease include but are not limited to Cas9 and Cpf1. Cas9 (formerly referred to as Cas5, Csn1, or Csx12) is a Class 2 Type II Cas endonuclease (Makarova et al. 2015, Nature Reviews Microbiology Vol. 13:1-15). A Cas9-gRNA complex recognizes a 3′ PAM sequence (NGG for the S. pyogenes Cas9) at the target site, permitting the spacer of the guide RNA to invade the double-stranded DNA target, and, if sufficient homology between the spacer and protospacer exists, generate a double-strand break cleavage. Cas9 endonucleases comprise RuvC and HNH domains that together produce double strand breaks, and separately can produce single strand breaks. For the S. pyogenes Cas9 endonuclease, the double-strand break leaves a blunt end. Cpf1 is a Clas 2 Type V Cas endonuclease, and comprises nuclease RuvC domain but lacks an HNH domain (Yamane et al., 2016, Cell 165:949-962). Cpf1 endonucleases create “sticky” overhang ends.

Some uses for Cas9-gRNA systems at a genomic target site include but are not limited to insertions, deletions, substitutions, or modifications of one or more nucleotides at the target site; modifying or replacing nucleotide sequences of interest (such as a regulatory elements); insertion of polynucleotides of interest; gene knock-out; gene-knock in; modification of splicing sites and/or introducing alternate splicing sites; modifications of nucleotide sequences encoding a protein of interest; amino acid and/or protein fusions; and gene silencing by expressing an inverted repeat into a gene of interest.

The process for editing a genomic sequence at a Cas9-gRNA double-strand-break site with a modification template generally comprises: providing a host cell with a Cas9-gRNA complex that recognizes a target sequence in the genome of the host cell and is able to induce a double-strand-break in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the double-strand break.

To facilitate optimal expression and nuclear localization for eukaryotic cells, the gene comprising the Cas endonuclease may be optimized as described in WO2016186953 published 24 Nov. 2016, and then delivered into cells as DNA expression cassettes. In some aspects, the Cas endonuclease is provided as a polypeptide. In some aspects, the Cas endonuclease is provided as a polynucleotide encoding a polypeptide. In some aspects, the guide RNA is provided as a DNA molecule encoding one or more RNA molecules. In some aspects, the guide RNA is provided as RNA or chemically-modified RNA. In some aspects, the Cas endonuclease protein and guide RNA are provided as a ribonucleoprotein complex (RNP).

Zinc Finger Mediated Genome Editing

As an example, the genetically modified cell or plant described herein, is generated using a zinc finger nuclease-mediated genome editing process. The process for editing a chromosomal sequence includes for example: (a) introducing into a cell at least one nucleic acid encoding a zinc finger nuclease that recognizes a target sequence in the chromosomal sequence and is able to cleave a site in the chromosomal sequence, and, optionally, (i) at least one donor polynucleotide that includes a sequence for integration flanked by an upstream sequence and a downstream sequence that exhibit substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. A zinc finger nuclease includes a DNA binding domain (i.e., zinc finger) and a cleavage domain (i.e., nuclease). The nucleic acid encoding a zinc finger nuclease may include DNA or RNA. Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Choo et al. (2000) Curr. Opin. Struct. Biol. 10:411-416; and Doyon et al. (2008) Nat. Biotechnol. 26:702-708; Santiago et al. (2008) Proc. Natl. Acad. Sci. USA 105:5809-5814; Urnov, et al., (2010) Nat Rev Genet. 11(9):636-46; and Shukla, et al., (2009) Nature 459 (7245):437-41. An engineered zinc finger binding domain may have a novel binding specificity compared to a naturally-occurring zinc finger protein. As an example, the algorithm of described in U.S. Pat. No. 6,453,242 may be used to design a zinc finger binding domain to target a preselected sequence. Nondegenerate recognition code tables may also be used to design a zinc finger binding domain to target a specific sequence (Sera et al. (2002) Biochemistry 41:7074-7081). Tools for identifying potential target sites in DNA sequences and designing zinc finger binding domains may be used (Mandell et al. (2006) Nuc. Acid Res. 34:W516-W523; Sander et al. (2007) Nuc. Acid Res. 35:W599-W605).

An exemplary zinc finger DNA binding domain recognizes and binds a sequence having at least about 80% sequence identity with the desired target sequence. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

A zinc finger nuclease also includes a cleavage domain. The cleavage domain portion of the zinc finger nucleases may be obtained from any endonuclease or exonuclease. Non-limiting examples of endonucleases from which a cleavage domain may be derived include, but are not limited to, restriction endonucleases and homing endonucleases. See, for example, 2010-2011 Catalog, New England Biolabs, Beverly, Mass.; and Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). One or more of these enzymes (or functional fragments thereof) may be used as a source of cleavage domains.

Meganuclease-based Genome Editing

Another example for genetically modifying the cell or plant described herein, is by using “custom” meganucleases produced to modify plant genomes (see e.g., WO 2009/114321; Gao et al. (2010) Plant Journal 1:176-187. The term “meganuclease” generally refers to a naturally-occurring homing endonuclease that binds double-stranded DNA at a recognition sequence that is greater than 12 base pairs and encompasses the corresponding intron insertion site. Naturally-occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease.

Naturally-occurring meganucleases, for example, from the LAGLIDADG family, have been used to effectively promote site-specific genome modification in plants, yeast, Drosophila, mammalian cells and mice. Engineered meganucleases such as , for example, LIG-34 meganucleases, which recognize and cut a 22 basepair DNA sequence found in the genome of Zea mays (maize) are known (see e.g., US 20110113509).

TAL Endonucleases (TALEN)

TAL (transcription activator-like) effectors from plant pathogenic Xanthomonas are important virulence factors that act as transcriptional activators in the plant cell nucleus, where they directly bind to DNA via a central domain of tandem repeats. A transcription activator-like (TAL) effector-DNA modifying enzymes (TALE or TALEN) are also used to engineer genetic changes. See e.g., US20110145940, Boch et al., (2009), Science 326(5959): 1509-12. Fusions of TAL effectors to the FokI nuclease provide TALENs that bind and cleave DNA at specific locations. Target specificity is determined by developing customized amino acid repeats in the TAL effectors.

Brassica Transformation Methods for Gene Editing

Methods are provided for transforming a Brassica plant with a construct to cause gene edits. The methods involve transforming a Brassica cell with a DNA construct by microprojectile bombardment. The methods further involve regenerating the transformed cell into a transformed Brassica plant. Such a transformed Brassica plant possesses at least one copy of the DNA construct, or portion thereof, incorporated into its genome. The transformed Brassica plants may be stably transformed. Such transformed Brassica plants are capable of producing at least one offspring that possesses at least one copy of the DNA construct, or portion thereof, stably incorporated within its genome.

Cells of the embodiments disclosed herein may originate from (1) pre-incubated microspores, (2) microspore-derived embryos or (3) microspore-derived hypocotyls. It is recognized that the cells of these tissues are most likely haploid. The cells may be diploid if the cells undergo spontaneous chromosome doubling, or if the cells are subjected to chromosome doubling agents. Transformation of haploid cells is advantageous because the resulting chromosome doubled transgenic plant is homozygous.

A DNA construct of interest is introduced into the cell by microprojectile bombardment. Microprojectile bombardment is also known by other terms, including particle bombardment, microparticle bombardment, ballistic particle acceleration and biolistic transformation. Generally, such methods involve applying to or coating the surface of microprojectiles with the DNA construct of interest, and then delivering the DNA-coated microprojectiles to the target tissue at a velocity sufficient to allow the particles to pass through cell walls and membranes and thus, enter plant cells. See, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental. Methods, ed. Gamborg and Phillips (Springer-Verlag, Berlin); and McCabe et al. (1988) Biotechnology 6:923-926.

The methods disclosed herein do not depend on a particular DNA construct. Any DNA construct that may be introduced into a cell by microprojectile bombardment may be employed. DNA constructs may comprise at least one nucleotide sequence of interest operably linked to a promoter that drives expression in a plant cell. DNA constructs may comprise a selectable marker gene and at least one additional nucleotide sequence of interest operably linked to a promoter that drives expression in a plant cell. Additionally, DNA constructs may comprise a selectable marker gene and at least one additional nucleotide sequence that is capable of conferring a desired trait on a Brassica plant.

The methods may additionally include regenerating the transformed cell into a stably transformed Brassica plant. Regeneration of the transformed plant involves culturing the transformed cell under conditions that result in the growth and development of the transformed cell into a transformed plant. The transformed cell or descendents thereof may develop into a transformed embryo, particularly a transformed microspore-derived embryo or somatic embryo which then develops into a transformed plant. Alternatively, the transformed cell and descendents thereof may develop into a transformed organ, such as, for example, an adventitious shoot. It is recognized that regenerating a transformed Brassica plant from a transformed cell via an adventitious shoot may additionally involve the formation of callus before adventitious shoot formation. Such an adventitious shoot may be used to produce the stably transformed Brassica plant. Such methods generally involve culturing an adventitious shoot in a medium and environment which favors the formation of adventitious roots on the adventitious shoot.

Methods for rooting adventitious shoots are available. The methods disclosed herein do not depend on a particular method for rooting transformed Brassica shoots. Any suitable method for rooting adventitious shoots may be employed. Generally, rooting adventitious shoots will involve incubating shoots, for a period of time, on a medium that contains an effective amount of an auxin, such as, for example, indolebutyric acid, to induce root formation. See, for example, Moloney et al. (1989) Plant Cell Reports 8:238-242 and Radke et al. (1992) Plant Cell Reports 11:499-505. Rooted shoots may then be removed from culture, transferred to soil or potting medium and subjected to environmental conditions that favour growth, maturation and seed production.

It is recognized that the transformed embryos, transformed adventitious organs, and transformed plants may be chimeric. That is, such transformed embryos, organs and plants may be comprised of both transformed and non-transformed cells, or may be comprised of two or more differentially transformed cells. It is further recognized that such chimeric plants may give rise to progeny plants that comprise a DNA construct of interest, or portion thereof, stably incorporated into the genomes of all of their somatic and germ line cells.

Certain methods involve the transformation of cells from Brassica plants. The cells may be haploid cells. While haploid cells generally do not give rise to diploid plants, it is recognized that occasionally a haploid cell may spontaneously give rise to a diploid cell that is capable of developing into a fertile plant. If necessary, chromosome-doubling agents may be employed to increase the ploidy of a haploid cell two fold. That is, a haploid cell becomes a diploid cell. Such a diploid cell may give rise to a fertile, stably transformed Brassica plant. The methods disclosed herein do not depend on a particular genetic mechanism of chromosome doubling. It is likely, however, that chromosome doubling results from chromosome duplication as would occur for example, during mitosis, but in the absence of cytokinesis.

Induced chromosome doubling generally involves administering an effective amount of a chromosome-doubling agent to a cell, preferably a haploid cell. Any agent that is known to increase the ploidy of cells may be employed. Chromosome-doubling agents include, but are not limited to, trifluralin, colchicine, oryzalin, amiprophosmethyl and pronamide. Depending on the desired outcome, a chromosome-doubling agent may be administered to a tissue, or a cell thereof, before, after, or both before and after, introducing a DNA construct into a cell by microprojectile bombardment. In certain methods, an effective amount of a chromosome-doubling agent is administered after bombardment.

The plants regenerated from transformed Brassica cells are referred to as the T0 generation or T0 plants. The seeds produced by various sexual crosses of T0 generation plants are referred to as T1 progeny or T1 generations. When T1 seeds are germinated, the resulting plants are also referred to as T1 generation. Seeds produced on the T1 plant or from crosses using T1 pollen, are referred to as T2 seeds, which give rise to T2 plants. Seeds produced on the T2 plant or from crosses using T2 pollen, are referred to as T3 seeds. T3 seeds give rise to T3 plants. Accordingly, the generations progress through T4, T5, T6, etc. The seeds and plants of the T1, T2, T3, T4, etc. can be analyzed to ensure successful transmission of the transgene. Various sexual crosses are possible. For example, the plants can be selfed, outcrossed or backcrossed. Alternatively, the transgenic plants (T0, T1, T2, etc) can be propagated asexually, for example by cloning, tissue culture, cuttings, microspore culture.

In an embodiment, methods are provided for transforming Brassica pre-incubated microspores and regenerating stably transformed plants therefrom. These methods generally involve bombarding pre-incubated microspores with microprojectiles coated with a DNA construct of interest. Microspores are isolated by methods that are available to those skilled in the art. The microspores are haploid. The microspores may be isolated and cultured in a medium with a high level of sucrose, for example 17% sucrose, for 2 to 3 days. The high level of sucrose is recommended to ensure the integrity of the microspores immediately after isolation. Further, high osmotic stress would have a positive effect on embryogenesis induction (Maraschin et al., 2005 J Exp Bot 56:1711-1726 and Prem et al. 2005 In Vitro Cell. Dev. Biol.-Plant 41:266-273). The microspores are then cultured for 4 to 8 days in medium containing a reduced level of sucrose, for example in the range of 10% sucrose to promote microspore division. Accordingly, a pre-incubation period of 2 to 10 days is suitable.

After the pre-incubation period, the pre-incubated microspores are collected in a manner to enrich for viable and embryogenic microspores. This can be done, for example, by using a commercially available synthetic nylon sieve of 15 to 48 μm in pore size. The pore size may be between 15 and 25 μm. The embryogenic microspores can also be enriched by commercially available gradient centrifugation. Commercially available colloidal silica concentration is between 35 to 45%. The sieve holding the pre-incubated microspores is then placed on an osmotic treatment medium prior to bombardment, during bombardment and for a period of time after bombardment. The osmotic treatment induces slight plasmolysis of the microspores to reduce the likelihood that they will not burst due to the bombardment procedure. During bombardment, a surface of the microspores may be exposed to the path of the bombarding particles coated with DNA to facilitate entry of the particles and also to prevent any sudden influx of medium (i.e. the surface of the microspores that is exposed is not embedded in the medium). The osmotic treatment facilitates this. For example, the pre-incubated microspores may be subjected to the osmotic treatment for 1 to 2 hours prior to bombardment, during the bombardment, and for 1 to 24 hours after bombardment. For example, the osmotic treatment may comprise placing the pre-incubated microspores on medium containing between 0.8 to 1.6% commercially available agar or an agar substitute, between 17 and 19% sucrose and 1 g/l MES (2-[N-Morpholino]ethanesulfonic acid). The osmotic treatment can also be done by placing microspores (optionally on sieves) in a petri dish (3.5 cm in diameter) prior to bombardment, during the bombardment and after the bombardment. A piece of filter paper (3.2 cm in diameter) wet with NLN-13S medium is placed in the petri dish to prevent microspore dehydration.

Following bombardment, the microspores and sieve can be cultured in medium containing a doubling agent and a high level of sucrose, for example 13% sucrose, for about 7 days. If the DNA construct of interest comprises a selectable marker gene, the bombarded pre-incubated microspores may be transferred to medium containing an appropriate selective agent for that particular selectable marker gene. Such a transfer may occur immediately after bombardment or after a period of time. For example, the transfer may occur between 0 and about 30 days after bombardment.

During the selection stage, the pre-incubated microspores may be sub-cultured in selection NLN medium, which may contain a reduced sucrose content, for example 6.5% and growth regulators, for example cytokinins and auxins. Selection may be conducted in the light. The pre-incubated microspores may then be monitored for the appearance of transformed embryos and/or adventitious shoots. Such transformed embryos and/or adventitious shoots may then be cultured in shoot regeneration medium that may contain MS or B5 components and further, may also contain selection agents (for example, kanamycin or glyphosate) with or without plant growth regulators. The regenerated shoots are rooted in B5 medium containing 0.1 mg/l GA3.

In another embodiment, methods are provided for transforming cells from microspore-derived embryos with microprojectiles coated with a DNA construct of interest. Methods are generally known for producing embryos from Brassica microspores. Like the microspores themselves, the cells comprising such microspore-derived embryos are haploid. In certain methods disclosed herein, whole microspore-derived embryos are bombarded with DNA-coated microprojectiles. The microspore-derived embryos may be greater than 10 days old and approximately greater than 1.5 mm in size. The microspore-derived embryos can be between 11 and 20 days old. The embryos may be globular or heart shaped. The embryos are placed on osmotic medium prior to, during and after bombardment for the same reasons as discussed above. At least one surface of the embryos should be exposed to the path of the bombarded particles (i.e. not in the medium) during bombardment to facilitate entry of the particle and to minimize sudden influx of medium into the cell. For example, the embryos may be subjected to the osmotic treatment for approximately 4 hours prior to bombardment and for approximately 20 hours (for example, overnight) after bombardment. The osmotic treatment may comprise, for example, a medium containing 17 to 19% sucrose and 1.5% agar, and acts to reduce the likelihood for the cells of the embryos from bursting during and after bombardment. Alternatively, the osmotic treatment may comprise placing the embryos on a petri dish having a wet filter paper. The embryos are then transferred to regeneration medium. The regeneration media may include, but are not limited to, B5 media, MS-based media (MS salts with organics, 2% (w/v) sucrose, 0.6% (w/v) Sigma agar, pH 5.8). Embryo-derived hypocotyls may be excised and cultured in selection medium to induce transgenic shoots. Typically, a microspore-derived embryo gives rise to a single or a few adventitious shoots as a result of growth from the apical meristem or hypocotyl area. Methods of the second and third embodiments involve adventitious shoot regeneration of the microspore-derived embryos and microspore-derived hypocotyls. Such methods find use in increasing the number of transformed plants recovered from a transformation attempt.

Adventitious shoot regeneration involves the formation of multiple shoots arising from a microspore-derived embryo. Thus, a single microspore-derived embryo can yield multiple transformed shoots from a transformation. Typically, all of the transformed shoots that arise from a single microspore-derived embryo are thought to be an independent transformant. That is, each transformed shoot is derived from an independently transformed cell and thus, is genetically distinct. For the purposes of this investigation, however, all multiple events from each embryo were combined.

Methods of adventitious shoot regeneration are generally known. While the methods of editing microspores do not depend on a particular method of adventitious shoot regeneration, the methods may involve subjecting the microspore-derived embryos to an effective amount of cytokinin to induce adventitious shoots. Adventitious shoot regeneration may be accomplished within less than about 30 days after administering a cytokinin to the microspore-derived embryos. Adventitious shoot regeneration may be accomplished within less than about 10 days after administering the cytokinin. The methods of secondary regeneration may additionally involve subjecting the microspore-derived embryos to an effective amount of an auxin. In exemplary methods, an effective amount of a cytokinin is administered, with or without an effective amount of an auxin, to the microspore-derived embryos following bombardment to induce adventitious shoot regeneration.

If the DNA construct utilized in methods of the second embodiment comprises a selectable marker gene, selection may be applied immediately after bombardment or after a period of time of less than 2 days to about 30 days. Selection may be applied by subjecting the microspore-derived embryos to an effective amount of an appropriate selective agent for the selectable marker gene of the DNA construct of interest. An effective amount of the selective agent may be added to the medium on which the microspore-derived embryo is cultured. The selective agent may be administered alone or in combination with one or more other compounds such as a chromosome-doubling agent or a plant growth regulator.

In another embodiment, methods are provided for particle bombardment of microspore-derived hypocotyls. Approximately 21 days after culture, when the embryos are generally torpedo shaped, the culture medium is diluted with fresh culture medium and the microspores are allowed to culture for approximately 5 more days. The majority of the embryos are generally at the cotyledon stage after 26 days in culture. Hypocotyl sections from the embryos are excised and cultured for a period of time on medium supplemented with plant growth regulators to induce cell division. For example, the excised hypocotyls may be cultured overnight on MMW+4 mg/l BAP+0.25 mg/l NAA.

Prior to bombardment, during bombardment and after bombardment, the excised hypocotyls are subjected to osmotic treatment as described above. In addition, a surface of the hypocotyls in direct line with the bombardment route is exposed to the path of the bombarding particle (i.e. the surface of the hypocotyls that is in the direct line with the bombardment route is not embedded in the medium) to facilitate entry of the particle coated with DNA and to avoid any sudden influx of medium. The osmotic medium may comprise 17 to 19% sucrose, 1.5% agar and 1 g/l MES. The hypocotyls may be cultured on the osmotic medium for 4 hours prior to bombardment and overnight after bombardment. After osmotic treatment, the hypocotyls may be transferred to bud induction medium (for example, MMW+4 mg/l BAP+25-100 mg/l KAN). A bud can be an immature shoot, leaf, embryo or flower. Hypocotyls comprising adventitious buds may then be transferred to shoot regeneration medium (for example, MMW without hormones, or MMW+0.2 mg/l BAP). A selection agent may be added to any of the media after bombardment.

Additionally, the methods of the first, second, and third embodiments may comprise administering an effective amount of a chromosome doubling agent to the culture medium before, or optionally after, bombardment. Adding a chromosome doubling agent is not necessary in all cases, because the rate of spontaneous doubling can be high, especially in the embodiments employing microspore-derived embryos and microspore-derived hypocotyls. Such chromosome-doubling agents and methods of use are known and were discussed above.

The methods disclosed herein involve the use of plant growth regulators such as, for example, auxins and cytokinins. The plant growth regulators may include, but are not limited to, both free and conjugated forms of naturally occurring plant growth regulators. Additionally, the plant growth regulators may encompass synthetic analogues and precursors of such naturally occurring plant growth regulators.

Naturally occurring and synthetic analogues of auxins include, but are not limited to, indoleacetic acid (IAA), 3-indolebutyric acid (IBA), a-napthaleneacetic acid (NAA), 2,4-dichlorophenoxyacetic acid (2,4-D), 4-(2,4-dichlorophenoxy) butyric acid, 2,4,5-trichlorophenoxyacetic acid (2,4,5-T), (4-chloro-2-methylphenoxy) acetic acid (MCPA), 4-(4-chloro-2-methylphenoxy) butanoic acid (MCPB), mecoprop, dicloprop, quinclorac, picloram, triclopyr, clopyralid, fluroxypyr and dicamba.

Naturally occurring and synthetic analogues of cytokinins include, but are not limited to, kinetin, thidiazuron (TDZ), zeatin, zeatin riboside, zeatin riboside phosphate, dihydrozeatin, isopentyl adenine and 6-benzyladenine (BAP).

The methods may include use of G418 disulfate salt (Gibco™), also sold as Geneticin™ from Fluka as a selection agent or glyphosate as a selection agent. After bombardment of pre-incubated microspores, selection may be done in liquid medium in dark first and then under low light intensity (for example, approximately 240 foot-candles or 2,583 lux). However, other selection agents, as is known to those skilled in the art, can be used. For example, kanamycin, and other herbicides, like Basta™ and Chlorsulfuron™.

Stable transgenic plants may be confirmed by polymerase chain reaction (PCR) analysis and Southern blot hybridization analysis. The DNA constructs also encompass all forms of nucleotide constructs including, but not limited to, single-stranded forms, double-stranded forms, hairpins, stem-and-loop structures and the like. Furthermore, it is recognized that the methods may employ a DNA construct that is capable of directing, in a transformed plant, the expression of at least one protein, or at least one RNA, such as, for example, an rRNA, a tRNA or an antisense RNA that is complementary to at least a portion of an mRNA. Typically such a DNA construct is comprised of a coding sequence for a protein or an RNA operably linked to 5′ and 3′ transcriptional regulatory regions. Alternatively, it is also recognized that some of the methods disclosed herein may employ a DNA construct that is not capable of directing, in a transformed plant, the expression of a protein or RNA.

In addition, it is recognized that methods do not depend on the incorporation of the entire DNA construct into the genome, only that the genome of the Brassica plant is altered as a result of the introduction of the DNA construct into a Brassica cell. For example, alterations to the genome include additions, deletions and substitution of nucleotides in the genome. While the methods do not depend on additions, deletions, or substitutions of any particular number of nucleotides, it is recognized that such additions, deletions or substitutions comprise at least one nucleotide.

The DNA constructs also encompass nucleotide constructs, that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism.

Additionally, the term “DNA-coated microprojectiles” used herein is not intended to limit the methods to microprojectiles coated with DNA. Rather, the term “DNA-coated microprojectiles” as used herein encompasses microprojectiles coated with any one or more of the DNA constructs as described herein. The term “RNA-coated microprojectiles” used herein is not intended to limit the methods to microprojectiles coated with RNA, for example, guide RNA. Rather, the term “RNA-coated microprojectiles” as used herein encompasses microprojectiles coated with any one or more of the RNAs e.g., guide RNA, crRNA, tracrRNA or constructs capable of transcribing those RNAs as described herein. Similarly, the term “RNP-coated microprojectiles” as used herein encompasses microprojectiles coated with any one or more of the RNAs e.g., guide RNA, crRNA, tracrRNA, Cas endonucleases or a combination thereof to the extent these complexes form a ribonucleoprotein complex on the microparticle or microprojectile.

The DNA constructs may be comprised of expression cassettes for expression in the Brassica plant of interest. The expression cassette may include 5′ and 3′ regulatory sequences operably linked to a gene of interest. By “operably linked” is intended a functional linkage between a regulatory sequence and a second sequence, wherein the regulatory sequence affects initiation and mediation of transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes. Such an expression cassette is provided with a plurality of restriction sites for insertion of the gene of interest sequence to be under the transcriptional regulation of the regulatory regions. The expression cassette may additionally contain selectable marker genes. The expression cassette may include in the 5′-3′ direction of transcription, a transcriptional and translational initiation region, a gene of interest and a transcriptional and translational termination region functional in plants. The transcriptional initiation region, the promoter, may be native (or analogous) or foreign (or heterologous) to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence. While it may be preferable to express the gene of interest using heterologous promoters, the native promoter sequences may be used. Such constructs would change expression levels of the gene of the interest in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

A number of promoters can be used. The promoters can be selected based on the desired outcome. The nucleic acids can be combined with constitutive, tissue-preferred, or other promoters for expression in Brassica plants. Tissue-preferred promoters can be utilized to target enhanced expression of the gene of interest within a particular plant tissue. Such promoters can be modified, if necessary, for weak expression.

“Seed-preferred” promoters include both “seed development” promoters (those promoters preferentially active during seed development such as promoters of seed storage proteins) as well as “seed-germinating” promoters (those promoters preferentially active during seed germination).

Various changes in phenotype are of interest including modifying the fatty acid composition in a plant, altering the amino acid content of a plant, altering a plant's pathogen defense mechanism, enhancing tolerance to abiotic stress, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes or nucleotide sequences of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered by methods in addition to using traditional breeding methods. Modifications include increasing content of oleic acid, saturated and unsaturated oils, increasing levels of lysine and sulfur, providing essential amino acids, and also modification of starch.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or Basta® (e.g., the bar gene), or other such genes known in the art. The ALS-gene mutants encode resistance to the herbicide chlorsulfuron (Swanson et al (1989) Theor Appl Genet 78:525-530, EP0257993 B1). The glyphosate acetyl transferase (GAT) gene confers resistance to glyphosate (Castle et al. (2004) Science 304:1151-1154).

Sterility genes can also be encoded in an expression cassette and provide an alternative to physical emasculation. Examples of genes used in such ways include male tissue-preferred genes and genes with male sterility phenotypes such as avidin and streptavidin, described in U.S. Pat. No. 5,962,769 (Albertsen et al., 1999) and Barnase (Block and Debrouwer (1993) Planta 189: 218-225) Other genes include kinases and those encoding compounds toxic to either male or female gametophytic development.

It is recognized that a DNA construct may comprise an antisense construction complementary to at least a portion of a messenger RNA (mRNA) of a gene of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% or more sequence identity to the complementary sequences may be used. Furthermore, portions of the antisense nucleotides may be used to disrupt the expression of the target gene. Generally, sequences of at least 50 nucleotides, 100 nucleotides, 200 nucleotides, or greater may be used. Typically, such antisense constructions will be operably linked to a promoter that drives expression in a plant.

The DNA constructs may also be employed in sense suppression methods to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, for example, greater than about 65%, 85% or 95% sequence identity. See, U.S. Pat. Nos. 5,283,184 and 5,034,323.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NPTII), and hygromycin phosphotransferase (HPT). Genes conferring resistance to herbicidal compounds may also be used, such as glyphosate acetyl transferase, glufosinate ammonium, bromoxynil, imidazolinones, and 2,4-dichlorophenoxyacetate (2,4-D). The above list of selectable marker genes and screenable marker genes is not meant to be limiting. Any selectable and/or screenable marker gene can be used.

Brassica plants include, but are not limited to, Brassica carnata (Ethiopian mustard), Brassica juncea (leaf mustard), Brassica napus (rape), Brassica napus var. rapifera (Swedish turnip), Brassica nigra (black mustard), Brassica oleracea, Brassica oleracea var. acephala (kale), Brassica oleracea var. alboglabra (Chinese kale), Brassica oleracea var. hotrytis (cauliflower, heading broccoli), Brassica oleracea var. capitata (cabbage), Brassica oleracea var. gemmifera (Brussel sprouts), Brassica oleracea var. gongylodes (Kohlrabi), Brassica rapa (field mustard; also known as Brassica campestris), Brassica rapa subsp. chinensis (bok-choy), and Brassica rapa subsp. pekinensis (Chinese cabbage).

In certain embodiments, the Brassica plants are oilseed Brassica plants. Such oilseed Brassica plants are used for oil production and include but are not limited to, Brassica juncea, Brassica napus and Brassica rapa. The Brassica plants may be canola plants. Such canola plants are selections of oilseed Brassica plants (Brassica rapa, Brassica napus and Brassica juncea) that contain low levels of both erucic acid and glucosinolates in their seeds. Canola oil must contain less than 2% erucic acid and the solid component of the seed must contain less than 30 micromoles of any one or any mixture of 3-butenyl glucosinolate, 4-pentenyl glucosinolate, 2-hydroxy-3 butenyl glucosinolate, and 2-hydroxy-4-pentenyl glucosinolate per gram of air-dry, oil free solid. The seeds of such canola plants are favored for the extraction of edible oils.

### Microspore-Derived Embryos—Experiments

Experiments 1 to 2 describe the work done using microspore-derived embryos as the target tissue. Microspores were isolated as is known to those skilled in the art, for example, see Swanson et al. 1987 and cultured in NLN medium (see Section entitled “Media Recipes” for components of all media used) for approximately 11 to 14 days. However, the microspore-derived embryos can be cultured for between 11 to 20 days. The embryos were produced from the microspores and were detectable with the naked eye. The size of the microspore-derived embryos was generally smaller than 1 mm and the embryos were globular or heart shaped.

The microspore-derived embryos were collected to enrich for viable embryos. For example, this may be done using a pipette and transferring the embryos to a filter paper or membrane, for example Gelman™ membrane (Prod. No. 60110). The filter paper or membrane can have a 0.8 μm pore size. The embryos and membrane were cultured on osmotic medium for example, the medium may contain 17 to 19% sucrose+0.8 to 1% agar+1 g/l MES, pH 6.0. The embryos were subjected to the osmotic treatment before, during and after bombardment. For example, the embryos were subjected to the osmotic treatment for 4 hours prior to bombardment. The DNA construct used in bombardments was PHP18644. PHP18644, and other vectors used are described in the Table 9. The bombardment was done as is known to those skilled in the art, using approximately 10 ng to 5 μg of DNA per preparation, 15 μg to 300 μg of gold particles of approximately 0.4-1.0 micron per shot, CaCl2 at 0.5M to 2.5M and a rupture disk of 650, 900 or 1100 psi. After bombardment, the embryos were cultured on the osmotic medium for 4 hours to 20 hours (approximately overnight) and then transferred to liquid NLN medium. The bombarded embryos were subsequently cultured for 7 to 14 days. The bombarded embryos or hypocotyls excised from the embryos at age 3 to 4 weeks were transferred from liquid NLN medium to solid selection medium MMW+IAA+TDZ+K25-50 (embryos) or MMW+BAP+K25-50 (hypocotyls) for bud induction. The resistant regenerated buds were excised and cultured on MMW-H+K50-100 for plant regeneration.

### Experiment 1

### Effect of Various Rupture Disks on GUS Transient Expression Using Microspore-Derived Embryos

The objective was to screen different rupture disks to determine those that would result in the highest transformation efficiency. Part A: Fourteen-day old microspore-derived embryos were pre-incubated for 4 hours on osmotic medium containing 17% sucrose, 1 g/l MES and 10 g/l agar with pH 6.0. The microspore-derived embryos were bombarded with PHP18644 precipitated on gold particles (100 μg/shot). The bombarded embryos were cultured for 4 hours after bombardment on the osmotic medium containing 17% sucrose, 1 g/l MES and 10 g/l agar with pH 6.0 and then cultured in NLN medium. Rupture disks of 450 psi, 650 psi and 900 psi were tested.

Transient transformation efficiency was determined by analyzing the bombarded embryos using the GUS assay, as is known to those skilled in the art. Table 1 shows the results of the GUS assay. Rupture disk 900 psi produced the greatest number of cells expressing GUS in this experiment.

TABLE 1
Effect of cultivar and rupture disk on GUS transient
expression using microspore-derived embryos

| | | # of | |
|---|---|---|---|
| | Rupture | bombarded | GUS dots/ |
| Rep # | disk | embryos | embryos |
| Rep 1 | 450 psi | 200 | 4 |
| | 650 psi | 193 | 5.7 |
| | 900 psi | 207 | 2.9 |
| Rep 2 | 450 psi | 107 | 7.5 |
| | 650 psi | 54 | 20.4 |
| | 900 psi | 51 | 31.4 |
| Rep 3 | 450 psi | 39 | 69.2 |
| | 650 psi | 39 | 76.9 |
| | 900 psi | 51 | 80.4 |

Part B: The construct used was PHP18644. Bombarded small embryos were cultured in NLN-13S for 2-3 weeks. The embryos or excised hypocotyls from the embryos were cultured in bud regeneration medium MMW+IAA+TDZ+kanamycin (50 mg/l) (embryos) or MMW+BAP+Kanamycin (50 mg/l) (for hypocotyls).

Table 2 shows the results of resistant shoot formation using different rupture disk strengths. In part B of the experiment, no significant difference was found using the 650 psi, 900 psi or 1100 psi rupture disks. The 650 and 900 psi rupture disks were easiest to use because less time was required to achieve the pressure to rupture the disks. Accordingly, the 650 psi and 900 psi rupture disks were used in later experiments. Table 2 also shows that excising hypocotyl segments from the bombarded embryos results in resistant shoot formation.

TABLE 2
Effect of rupture disk on resistant bud production
from bombarded microspore-derived embryos

| | Rupture | # hypocotyls or | Resistant |
|---|---|---|---|
| Rep # | disks (psi) | # Embryos | buds (%) |
| Rep 1 | 650 | 300 (E) | 0 (0.0) |
| | 900 | 350 (E) | 0 (0.0) |
| | 1100 | 450 (E) | 1 (0.2) |
| Rep 2 | 650 | 150 (H) | 2 (1.3) |
| | 1100 | 150 (H) | 0 (0.0) |
| Rep 3 | 650 | 400 (E) | 0 (0.0) |
| | 900 | 400 (E) | 1 (0.3) |
| | 1100 | 400 (E) | 0 (0.0) |
| Rep 4 | 650 | 850 (H) | 5 (0.6) |
| | 900 | 525 (H) | 3 (0.6) |
| | 1100 | 775 (H) | 3 (0.4) |

### Experiment 2

### Resistant Shoot Production from Bombarded Microspore-Derived Embryos in Solid Kanamycin Selection Medium and Confirmation of the Kanamycin Resistant Plants by Polymerase Chain Reaction (PCR) Analysis

The objective was to demonstrate that kanamycin resistant plants were produced from bombarded microspore-derived embryos. The construct used was PHP 18644 and the cultivar was 46A65. Kanamycin resistant shoots were produced by bombardment of microspore-derived embryos. The shoots were regenerated into kanamycin resistant plants. The kanamycin resistant plants were analyzed using the REDExtract-N-AMP™ plant PCR kit from Sigma as is known to those skilled in the art. DNA was extracted from 14 kanamycin resistant plants and analyzed by PCR for the nptll gene. Table 3 indicates that the nptll gene was found in 12 of the 14 plants. Accordingly, stable transgenic plants were obtained by bombarding microspore-derived embryos.

TABLE 3
Detection of nptII gene in kanamycin resistant plants
regenerated from bombarded microspore-derived embryos

| | | PCR-nptII |
|---|---|---|
| Rep | # Events | positive |
| Rep 1 | 9 | 7 |
| Rep 2 | 5 | 5 |
| Total | 14 | 12 |

### Microspore-Derived Hypocotyls:

Experiments 3 to 7 describe work done using microspore-derived hypocotyls. Microspores were isolated as is known to those skilled in the art and cultured in NLN medium for 21 to 28 days (Swanson et al., 1987). Hypocotyls were excised from the embryos produced from the microspores when the embryos were approximately 3-5 mm in size. Microspores of cultivar 46A65 were cultured in NLN medium for approximately 21 days. On the 21st day, the NLN medium was changed and diluted (1:20) with fresh NLN medium and the embryos were cultured for 5 more days. The hypocotyls were excised from the embryos and preconditioned overnight on MMW+BAP (4 mg/l)+NAA (0.25 mg/l). The hypocotyls were transferred to osmotic medium (for example, 17% sucrose+1% agar+1 g/l MES, pH 6.0) for 4 hours and then bombarded. The DNA construct used in bombardments was PHP 18644. After bombardment, the hypocotyls were cultured on the osmotic medium for approximately between 4 and 20 hours (for example, overnight). The bombarded hypocotyls were then cultured on MMW+BAP (4 mg/l)+kanamycin (25-50 mg/l) for bud induction. Regenerated buds were cultured on MMW+kanamycin (50-100 mg/l) or MMW+BAP (0.2 mg/l)+kanamycin (50-100 mg/l) for plant regeneration. The following experiments were done to test the effect of sucrose concentration, amount of gold particle used and the effect of rupture disk on transformation frequency.

### Experiment 3

### Effect of Sucrose Concentration in Osmotic Medium on Transient GUS Expression on Microspore-Derived Hypocotyls

The objective was to find the appropriate concentration of sucrose in the osmotic medium (sucrose+1% agar+1 g/l MES, pH 6.0) in order to produce the highest transformation efficiency. Sucrose concentrations of 15, 17, 19 and 21% were tested as shown in Table 4. The results indicated that using 19% sucrose produced the greatest number of transiently transformed cells.

TABLE 4
Effect of sucrose concentration in osmotic medium on transient
GUS expression in microspore-derived hypocotyls

| Sucrose | # | # with | # total | # spots/ |
|---|---|---|---|---|
| (%) | hypocotyls | GUS | spots | hypocotyls |
| 15 | 24 | 23 | 236 | 9.8 |
| 17 | 24 | 23 | 242 | 10.1 |
| 19 | 24 | 23 | 475 | 19.8 |
| 21 | 24 | 24 | 400 | 16.7 |

### Experiment 4

### Determining the Effect of the Amount of Gold Used per Shot on Transient GUS Expression in Microspore-Derived Hypocotyls

The objective was to determine the optimal amount of gold particles per bombardment. The construct used was PHP18644. Table 5 shows the results using 100, 200 and 300 μg of gold per shot. The results were not consistent in three experiments. 100 μg gold particles per shot were used in the remainder of the experiments.

TABLE 5
Effect of the amount of gold particle per bombardment on GUS
transient expression using microspore-derived hypocotyls

| | | | # with | |
|---|---|---|---|---|
| | Gold amount/ | # | GUS | Spots/per |
| Rep # | shot (ug) | hypocotyls | spots | hypocotyls |
| 1 | 100 | 32 | 1 | 0.1 |
| | 200 | 32 | 4 | 0.5 |
| | 300 | 33 | 4 | 1.3 |
| 2 | 100 | 24 | 24 | 39.6 |
| | 200 | 24 | 24 | 29.6 |
| | 300 | 25 | 25 | 35.4 |
| 3 | 100 | 24 | 21 | 20.1 |
| | 200 | 24 | 24 | 26.8 |
| | 300 | 24 | 24 | 40.1 |

### Experiment 5

### Effect of Rupture Disks on GUS Transient Expression on Microspore-Derived Hypocotyls

The objective was to determine which of four rupture disk strengths tested produced the greatest number of cells expressing GUS. The construct used was PHP18644. Table 6 shows the results of GUS transient expression using a rupture disk strength of 450 psi, 650 psi, 900 psi or 1100 psi. Results indicate that using rupture disks 650 psi and 900 psi produced the highest number of cells transiently expressing GUS.

TABLE 6
Effect of rupture disk strength on GUS transient
expression using microspore-derived hypocotyls

| | | | # with | |
|---|---|---|---|---|
| | Rupture | # | GUS | # GUS spots/ |
| Rep | disks | hypocotyls | spots | hypocotyls |
| 1 | 450 | 24 | 23 | 19.5 |
| | 650 | 24 | 24 | 48.3 |
| | 900 | 25 | 24 | 37.4 |
| | 1100 | 24 | 22 | 27.5 |
| 2 | 650 | 24 | 24 | 60.7 |
| | 900 | 25 | 25 | 60.5 |
| | 1100 | 25 | 25 | 49.4 |

### Experiment 6 Second Experiment to Determine the Effect of Rupture Disk on Resistant Bud Production Using Microspore-Derived Hypocotyls

The objective was to screen rupture disks to determine those that produced the greatest number of resistant buds on 50 mg/l kanamycin. The construct used was PHP18644. Table 7 shows the results using rupture disks of 450 psi, 650 psi and 900 psi. No significant difference was found between rupture disks 450 psi, 650 psi and 900 psi in this experiment.

TABLE 7
Effect of rupture disk strength on the production
of kanamycin resistant shoots using microspore-
derived hypocotyls as the bombardment tissue

| | Rupture | # | # with |
|---|---|---|---|
| Rep # | disks | hypocotyls | green buds |
| 1 | 450 psi | 375 | 6 (1.6%) |
| | 650 psi | 525 | 8 (1.5%) |
| | 900 psi | 300 | 5 (1.7%) |
| 2 | 450 psi | 200 | 1 (0.5%) |
| | 650 psi | 225 | 1 (0.4%) |
| | 900 psi | 100 | 1 (1.0%) |
| 3 | 450 psi | 300 | 2 (0.7%) |
| | 650 psi | 300 | 1 (0.3%) |
| | 900 psi | 300 | 2 (0.7%) |
| 4 | 450 psi | 300 | 0 (0.0%) |
| | 650 psi | 250 | 0 (0.0%) |
| | 900 psi | 300 | 3 (1.0%) |
| 5 | 450 psi | 150 | 1 (0.7%) |
| | 650 psi | 275 | 5 (1.8%) |
| | 900 psi | 200 | 3 (1.5%) |
| 6 | 450 psi | 125 | 0 (0.0%) |
| | 650 psi | 75 | 0 (0.0%) |
| | 900 psi | 25 | 1 (4.0%) |

### Experiment 7 Results of the GUS Assay on the Kanamycin Resistant Plants

The objective was to confirm the transgenic status of the kanamycin resistant plants by GUS analysis. Table 8 shows that seven kanamycin resistant plants were analyzed by GUS assay. Six plants were positive. This confirms that stable transgenic plants were produced by bombarding microspore-derived hytocotyls.

TABLE 8
Confirmation of transgenic status of kanamycin resistant
plants from microspore-derived hypocotyls by GUS analysis

| | Resistant | GUS Positive |
|---|---|---|
| Rep # | Plants | Plants |
| Rep 1 | 2 | 2 |
| Rep 2 | 3 | 2 |
| Rep 3 | 1 | 1 |
| Rep 4 | 1 | 1 |

### Pre-Incubated Microspores:

Experiments 8 to 17 describe the work done using pre-incubated microspores. The microspores were cultured in NLN-17S/10S for 2-10 days. Optimally, the microspores are cultured for 5-7 days. Embryos could not be detected with the naked eye. The embryogenic microspores were collected so that they remain viable and embryogenic with a Nitex™ sieve (15-36 um in pore size) or Percoll® (35-45%) gradient centrifugation. Pre-incubated microspores were used as bombarded materials. Any Brassica line that is capable of regenerating by microspore culture can be used. The microspores were cultured for 1-3 days in NLN-17S at 31.5° C., and then in NLN-10S for 4-5 days at 25° C. The constructs used in the bombardments were PHP18644, PHP21965, PHP22024, PHP22021, and PHP23560 (see Table 9). The constructs can be the full plasmid or the expression cassette only. For example, PHP22024 can be either the expression cassette or the full plasmid. The pre-incubated microspores were filtrated with sieves of pore size of 15 μm to 36 μm. The collected microspores were used for bombardment. The microspores were loaded on a sieve 15 μm or 20 μm on two layers of filter-paper and dried for less than one minute. The microspores and sieves were transferred to osmotic medium that contained B5 components, 1 g/l MES and 0.8-1.6% gelrite, 15-21% sucrose (pH 6.0). The microspores were treated for at least one hour on the osmotic medium before bombardment. The pre-incubated microspores were bombarded with 12.5 ng to 5 μg DNA per preparation, 15-100 μg Au particles per shot, 2.5 M CaCl2, and 650-900 psi rupture disk. During bombardment a surface of the pre-incubated microspores was exposed to the path of the bombarded particles coated with DNA (i.e. the surface was not embedded in the medium). The bombarded microspores were cultured at least four hours in the osmotic medium after bombardment. One to two sieves holding the microspores were cultured in 5 ml of NLN-13S with or without glyphosate per plate for approximately 7 days in the dark. After the 7 days, the spent medium was replaced with 10 ml of NLN-6.5S in each plate or the spent medium was diluted to obtain a sucrose concentration 6.5% in each plate. If the selectable marker gene was the NPTII gene, NAA, BAP and G418 were added to the medium. If the selectable marker gene was GAT, glyphosate was added in the medium. In this medium containing 6.5% sucrose, the embryos were cultured under dim light of approximately 240 foot-candles or 2,583 lux. The final concentration of G418 was 10 mg/l. and the concentration of glyphosate was 0.1 mM or 0.2 mM. A doubling agent was optionally added to the medium. Resistant embryos were recorded after two to three weeks of culture.

PHP 18644 and GAT (glyphosate acetyltransferase) constructs (see Table 9) were used in transformation experiments using pre-incubated microspores. The GAT gene was isolated from a bacterium as described by Castle et al. (2004) Science 304: 1151-1154. The gene was shuffled 11 rounds for increasing expression level of glyphosate acetyltransferase. There was one to several variants in each shuffling round. PHP18644 also contains the GUS marker gene. The constructs are described in Table 9.

TABLE 9
Constructs

| Constructs | Promoter | Selectable Marker | Variants and rounds |
|---|---|---|---|
| PHP18644 | CaMV | NPT11 | NA |
| PHP22024 | H2b | GAT | GAT4604, R 8 |
| PHP21965 | SCP1 | GAT | GAT4604, R 8 |
| PHP22021 | H2b | GAT | GAT4618, R 11 |
| PHP23560 | SCP1 | GAT | GAT4621, R 11 |

For a detailed description of the protocol, see section labeled “Protocol” following the Examples.

### Experiment 8 Development of a Selection Kill Curve Using Geneticin (G418) (Sigma G8168)

The objective was to determine the concentration of G418 that kills non-transgenic microspores. Prior to this experiment, selection was done on solid medium, not in liquid medium. Using liquid selection for Brassica transgenic cells is novel. Selection in liquid medium is advantageous for at least the following reasons: (a) it allows selection at an early stage, thereby eliminating the need for subsequent transfers of explants, (b) it allows for a cleaner selection because the explants are generally smaller when they are in liquid medium and the liquid allows all the surfaces of the explant to be exposed to the selection agent, (c) it may reduce the frequency of chimeras, and (d) a lower amount of the selection agent is needed therefore reducing the toxicity to the researcher and the environment. The effect of various concentrations of kanamycin on embryo growth in liquid medium was tested. The result showed that 10 mg/l kanamycin was sufficient to bleach non-bombarded embryos and inhibit non-bombarded embryo growth. However, kanamycin at 10 and 20 mg/l failed to inhibit the growth of the bombarded embryos, although the embryos were pale green and purple in color. Accordingly, it was difficult to discriminate transgenic tissue or embryos from non-transgenic. Replacing kanamycin with 5-10 mg/l G418 in NLN with 6.5% sucrose medium, resulted in a cleaner selection. Table 10 shows that 5 to 10 mg/l G418 is sufficient to differentiate transformed cells from untransformed cells.

TABLE 10
Effect of G418 on embryo growth and survival

| Conc. Of | | # Green embryos or | |
|---|---|---|---|
| G418 (mg/l) | # embryos | with green island | |
| 0 | 30 | 30 | |
| | 30 | 30 | |
| 1.25 | 30 | 30 | (paler) |
| | 28 | 28 | (paler) |
| 2.5 | 25 | 14 | (light green, smaller size) |
| | 25 | 11 | (light green, smaller size) |

| | | |
|---|---|---|
| 5 | 25 | 0 |
| | 25 | 0 |
| 10 | 30 | 0 |
| | 30 | 0 |

### Experiment 9 Production of G418 and Glyphosate Resistant Tissue and Buds After Bombardment of Pre-Incubated Microspores Followed by Selection in Liquid Medium Containing G418 or Glyphosate

The objective was to obtain transgenic plants by bombarding pre-incubated microspores that were cultured for up to 11 days and selecting resistant tissue and buds on liquid medium supplemented with 10 mg/I G418 or glyphosate at 0.1 mM and 0.2 mM.

In a first set of experiments, seven, eight, nine and eleven day old pre-incubated microspores were bombarded with PHP18644. NAA (0.5 mg/l) and BAP (0.05 mg/l), both growth regulators, were added to the selection medium comprising 10 mg/I G418 for enhanced cell growth conditions. Resistant microspores were transferred and cultured in the selection medium MMW+IAA+TDZ+AgNO35+C+K100 to induce resistant buds and confirm resistance. Accordingly, selection was initiated in the liquid culture medium with G418 and completed in the solid culture medium using kanamycin.

In a second set of experiments, 3, 4 and 5 day old pre-incubated microspores were bombarded with PHP23560. Glyphosate was added to the liquid and solid medium for selection.

Table 11 shows the results of the experiments. The data indicate that bombardment of pre-incubated microspores followed by selection in liquid medium using G418 or glyphosate produces resistant buds and PCR positive shoots.

TABLE 11
Effect of the pre-incubation period on resistant bud production

| | # days | | | PCR |
|---|---|---|---|---|
| | of | | Resistant | confirmed |
| Expt # | culture | Embryos | buds (%) | shoots |
| 1 | 9 | 2399 | 0 (0.0) | NA |
| 2 | 11 | 5629 | 0 (0.0) | NA |
| 3 | 8 | 1297 | 4 (0.3) | NA |
| 4 | 7 | 1998 | 4 (0.2) | NA |
| 5 | 5 | NA | NA | 6 |
| 6 | 4 | NA | NA | 3 |
| 7 | 3 | NA | NA | 3 |

### Experiment 10. Determining the Effect of Osmotic Culture Medium After Bombardment on Resistant Embryo Production

The objective was to determine whether culturing bombarded tissue on osmotic medium after bombardment would increase transformation efficiency. Table 12 indicates that bombarded pre-incubated microspores cultured on osmotic medium for 4 hours after bombardment produced a greater number of transgenic sectors than bombarded pre-incubated microspores that were not cultured on osmotic medium soon after bombardment. After the osmotic treatment, the bombarded pre-incubated microspores were cultured on liquid NLN medium.

TABLE 12
Effect of osmotic culture after bombardment
on resistant embryo production

| Treatments | # of embryos | # with green islands |
|---|---|---|
| Culture 0 hour | 2329 | 0 (0.0%) |
| after bombard | | |
| Culture 4 hour | 5057 | 80 (1.6%) |
| after bombard | | |

### Experiment 11. Determining the Efficiency of Resistant Bud Production from Bombarded Pre-Incubated Microspores Selected on Liquid Medium Supplemented with G418 at 10 mg/l and Confirmation of Resistant Plants by PCR Analysis

The objective was to confirm that selection in liquid medium produces resistant buds. The construct used was PHP 18644. Embryos that were resistant in the liquid selection medium were cultured in MMW+IAA (0.254 mg/l)+TDZ (1 mg/l)+AgNO3 (5 mg/l). Resistant buds were isolated and cultured in B5+GA+kanamycin (100 mg/l or MMW+kanamycin (100 mg/l) to produce shoots. Table 13 shows that resistant buds were regenerated from the embryos with sectors of resistance. Accordingly, selection in G418 liquid medium is efficient.

TABLE 13
G418 liquid medium selection efficiency
in terms of resistant bud production.

| | # green embryos | # embryos with |
|---|---|---|
| Rep # | or islands | resistant buds |
| Rep 1 | 51 | 40 |
| Rep 2 | 25 | 17 |
| Rep 3 | 1 | 1 |
| Rep 4 | 26 | 11 |

The nptll gene was confirmed in resistant plants using PCR analysis. Eight plants were selected at random and analyzed, six were found to have the nptll gene (Table 14).

TABLE 14
PCR analysis of resistant plants

| Reps | Resistant Plants | PCR-nptII positive |
|---|---|---|
| Rep 1 | 1 | 1 |
| Rep 2 | 1 | 1 |
| Rep 3 | 2 | 1 |
| Rep 4 | 4 | 3 |
| Total | 8 | 6 |

### Experiment 12. Glyphosate Resistant Embryo Selection After Bombarding Pre-Incubated Microspores with PHP22021 and PHP22024 and Followed by Selection in Liquid Medium NLN-6.5S Containing 0.1 mM and 0.2 mM Glyphosate

The objective was to compare the effect of glyphosate concentration on selection efficiency. Table 15 shows that using either 0.1 mM or 0.2 mM glyphosate allowed identification of resistant embryos. Both normal and abnormal embryos were produced. Although transgenics were identified using both 0.1 mM and 0.2 mM glyphosate, selection at 0.2 mM produced fewer false positive results. It was more efficient and eliminated additional transfers.

TABLE 15
Effect of the concentration of glyphosate
on resistant embryo selection

| | | | | # embryos | # embryos |
|---|---|---|---|---|---|
| | | | | (normal)/ | (abnormal)/ |
| Plasmid | Rep # | Glyphosate | # plates | plate | plate |
| PHP22021 | 1 | 0.1 mM | 12 | 3 | 23.8 |
| PHP22021 | 2 | 0.1 mM | 6 | 1.1 | 1 |
| | | 0.2 mM | 6 | 0 | 0 |
| PHP22021 | 3 | 0.1 mM | 9 | 0.6 | 0.2 |
| | | 0.2 mM | 9 | 0.1 | 0 |
| PHP22024 | 4 | 0.1 mM | 8 | 9.8 | 8.4 |
| | | 0.2 mM | 8 | 0.9 | 1.4 |
| PHP22024 | 5 | 0.1 mM | 7 | 2.1 | 2.4 |
| | | 0.2 mM | 6 | 0 | 0 |
| PHP22024 | 6 | 0.1 mM | 8 | 3.4 | 5.9 |
| | | 0.2 mM | 8 | 1.5 | 3.9 |

### Experiment 13. Determining the Effect of Sucrose Concentration in the Osmotic Medium on Resistant Embryo Production

The purpose of this experiment was to find the optimal concentration of sucrose in the osmotic medium. Microspores were cultured for 4-7 days using 17S/10S protocol. The construct used was PHP23560 and the concentration of DNA was 28 ng/preparation. The rupture disk was 900 psi. The bombarded microspores were cultured in the dark for 7-10 days in NLN-13S containing a doubling agent and 0.2 mM Glyphosate. The culture was diluted with NLN-OS without glyphosate to NLN-6.5S containing 0.1 mM glyphosate. The diluted cultures were incubated in the light for 2-3 weeks. There was no significant difference in the number of green embryos (normal and abnormal) produced using 15%, 17% and 19% sucrose.

The experiment was repeated as shown in Table 16. The results of this experiment showed that a sucrose concentration of 17% produced similar result as a sucrose concentration of 19% in four experiments.

TABLE 16
Effect of sucrose concentration in the osmotic
medium on resistant embryo production

| | | | Resistant |
|---|---|---|---|
| | Sucrose | | embryos/ |
| Rep # | concentration | # of plates | plate |
| 1 | 17% | 8 | 2.3 |
| | 19% | 8 | 1.8 |
| 2 | 17% | 1 | 0 |
| | 19% | 6 | 1 |
| 3 | 17% | 6 | 2.7 |
| | 19% | 6 | 1.8 |
| 4 | 17% | 7 | 5.1 |
| | 19% | 8 | 3.9 |
| Total | 17% | | 2.5 |
| | 19% | | 2.1 |

### Experiment 14. Determining the Effect of the Duration of the Osmotic Treatment on Resistant Embryo Production

The objective was to determine whether a 4 hour or 20 hour culture in osmotic medium results in a greater number of transgenic events. The construct used was PHP21965. Microspores were pre-incubated for 6 to 7 days, and then collected with 25 uM Nitex™ sieve. The collected microspores were subsequently loaded onto Nitex™ sieves with pore size of 20 uM. The bombarded microspores and Nitex™ sieves were cultured for 4 hours or 20 hours on osmotic medium B5+1 g/l MES+190 g/l sucrose+12 g/l gelrite (pH 5.8-6.0) after bombardments. Table 17 shows that treating microspores for 4 hours produced a comparable number of resistant embryos to treating microspores for 20 hours.

TABLE 17
Effect of the duration of the osmotic treatment
on resistant embryo production

| | Osmotic | | # | # resistant |
|---|---|---|---|---|
| Rep # | duration | | bombardments | embyros |
| 1 | 4 | h | 8 | 7 |
| | 44 | h | 4 | 1 |
| 2 | 4 | h | 8 | 8 |
| | 20 | h | 8 | 11 |
| 3 | 4 | h | 8 | 20 |
| | 20 | h | 8 | 4 |
| 4 | 4 | h | 8 | 7 |
| | 20 | h | 8 | 10 |
| 5 | 4 | h | 8 | 27 |
| | 20 | h | 8 | 40 |
| Summary | 4 | h | 41 | 69 (1.7/bombardment) |

| | | |
|---|---|---|
| 20 h (including | 37 | 66 (1.8/bombardment) |
| 44 h) | | |

### Experiment 15. Determining the Effect of the Rupture Disk Strength on Resistant Embryo Production

The purpose of this experiment was to compare effect of 650 psi and 900 psi rapture disk strength on resistant embryo production. Construct PHP23560 was used for bombardments. A total of nine experiments were conducted to compare rupture disks of 650 psi and 900 psi strength. No significant difference was found between rupture disks of 650 psi and 900 psi strength (Table 18). The rupture disk 650 psi was easier to use than the 900 psi rupture disk because less time was needed to build up helium pressure.

TABLE 18
Effect of rupture disk strength on resistant embryo production

| | Rupture | # | # resistant |
|---|---|---|---|
| Rep # | disks | bombardments | embryos |
| 1 | 650 | 8 | 21 |
| | 900 | 8 | 17 |
| 2 | 650 | 9 | 1 |
| | 900 | 4 | 5 |
| 3 | 650 | 8 | 10 |
| | 900 | 7 | 20 |
| 4 | 650 | 8 | 16 |
| | 900 | 8 | 13 |
| 5 | 650 | 8 | 8 |
| | 900 | 8 | 5 |
| 6 | 650 | 8 | 6 |
| | 900 | 8 | 3 |
| 7 | 650 | 8 | 0 |
| | 900 | 8 | 1 |
| 8 | 650 | 8 | 1 |
| | 900 | 8 | 3 |
| 9 | 650 | 7 | 4 |
| | 900 | 6 | 2 |
| Summary | 650 | 72 | 67 (0.93/bombardment) |
| | 900 | 65 | 69 (1.06/bombardment) |

### Experiment 16. PCR Analysis to Confirm Transgenic Plant Production via Bombarding Pre-Incubated Microspores

One hundred twenty-five resistant plants were confirmed positive using PCR analysis in 33 transformation experiments. Each experiment, on average, produced 3.8 transgenic plants (Table 19). The data for PHP22024 and PHP22021 includes data produced using both the DNA expression cassette and the entire plasmid. For example, of the 13 experiments listed under PHP22024, 9 were done using the plasmid and 4 were done using the expression cassette. The number of GAT positive plants was 55 and 34 respectively.

TABLE 19

| | # | Plants | # GAT |
|---|---|---|---|
| Constructs | Experiments | analyzed | positive |
| PHP22024 | 13 | 89 | 89 |
| PHP21965 | 3 | 8 | 7 |
| PHP22021 | 17 | 31 | 29 |
| Total | 33 | 128 | 125 |

### Experiment 17. Transgene Copy Number Analysis in GAT Transgenic Plants

Transgenic (T1) plants identified by glyphosate resistance were analyzed using Southern blot hybridization analysis. Plant genomic DNA was extracted using cetyltrimethylammonium bromide (CTAB) buffer (Rogers et al., (1994) Plant Molecular Biology Manual, 2nd Ed. 1:1-8.). The DNA samples were digested with Bam HI or Pst I. The hybridization probe was the GAT gene. The hybridization was made following Rajasekaran et al. (2000) Plant Cell Rep. 19: 539-545. The GAT gene copy number was determined by the highest number of bands from the hybridization blots for each digest. For example, if only one band was produced from both Bam HI and Pst I blots for an event, it indicated that the event had one copy of the GAT gene. Results from 42 events showed 17 events or 40% had only one copy of the GAT gene (Table 20).

TABLE 20
Transgenic copy number analysis in GAT transgenic plants

| | | Copy number of the GAT gene | | | |
|---|---|---|---|---|---|
| | | determined by Southern blot | | | |
| | Total number | hybridization analysis | | | |
| Constructs | of events | 1 | 2 | 3 | >3 |
| PHP22021 | 11 | 8 | 1 | 0 | 2 |
| PHP22024 | 29 | 8 | 11 | 7 | 3 |
| PHP21965 | 2 | 1 | 1 | 0 | 0 |
| Total | 42 | 17 (40%) | 13 (31%) | 7 (17%) | 5 (12%) |

### EXAMPLE 1

### Guide RNA/Cas endonuclease construct for genome modification for FAD2 and FAD3 in Brassica napus

To use the guide RNA/Cas endonuclease system in dicot plants, the Cas9 gene from Streptococcus pyogenes M1 GAS (SF370) was soybean optimized per standard techniques. To facilitate nuclear localization of the Cas9 protein in soybean cells, Simian virus 40 (SV40) monopartite nuclear localization signal was incorporated at the carboxyl-terminal of the Cas9 open reading frame. The Cas9 gene was operably linked to a soybean constitutive promoter such as the strong soybean constitutive promoter GM-EF1A2 (US Pub. 20090133159; SEQ ID NO: 3) by standard molecular biological techniques.

The second component used form a functional guide RNA/Cas endonuclease system for genome engineering applications is a duplex of the crRNA and tracrRNA molecules or a synthetic fusing of the crRNA and tracrRNA molecules, a guide RNA. To confer efficient guide RNA expression (or expression of the duplexed crRNA and tracrRNA) in dicots, the soybean U6 polymerase III promoter and U6 polymerase III terminator were used. Plant U6 RNA polymerase III promoters have been cloned and characterized from such as Arabidopsis and Medicago truncatula (Waibel and Filipowicz, NAR 18:3451-3458 (1990); Li et al., J. Integrat. Plant Biol. 49:222-229 (2007); Kim and Nam, Plant Mol. Biol. Rep. 31:581-593 (2013); Wang et al., RNA 14:903-913 (2008)). Soybean U6 small nuclear RNA (snRNA) genes were identified herein by searching public soybean variety Williams82 genomic sequence using Arabidopsis U6 gene coding sequence. Approximately 0.5 kb genomic DNA sequence upstream of the first G nucleotide of a U6 gene was selected to be used as a RNA polymerase III promoter for example, GM-U6-13.1 promoter, to express guide RNA to direct Cas9 nuclease to designated genomic site. The guide RNA coding sequence was 76 bp long and comprised a 20 bp variable targeting domain from a chosen soybean genomic target site on the 5′ end and a tract of 4 or more T residues as a transcription terminator on the 3′ end. The first nucleotide of the 20 bp variable targeting domain was a G residue to be used by RNA polymerase III for transcription. Other soybean U6 homologous genes promoters were similarly cloned and used for small RNA expression.

Cas9 endonuclease and guide RNA expression cassettes were linked into a single DNA construct. One guide RNA/Cas9 endonuclease target site (BNA-FAD2 CR1) was designed to target the FAD2 gene in Brassica napus. The BNA-FAD3 CR1 site was also designed to target the FAD3 gene in Brassica napus. The soybean U6 small nuclear RNA promoter, GM-U6-13.1, was used to express guide RNAs to direct Cas9 nuclease to designated genomic target sites. A soybean optimized Cas9 endonuclease expression cassette and a guide RNA expression cassette were linked in the plasmid (RTW1570 or RTW1572). For examples, the RTW1572 construct, which contained the BNA-FAD2 CR1 gRNA expression cassette and the Cas9 expression cassette, was made to target the FAD2 gene. Similarly, the RTW1750 construct, was made to target the FAD3 gene.

### Canola Genome Edited Plant Production

Canola microspores are cultured in liquid medium for 2-3 days without plant growth regulators and then bombarded with genome editing-related DNA using the transformation procedures disclosed above. The bombarded microspores divide and produce embryos through direct embryogenesis. A chromosome doubling reagent and selection reagent are included in the liquid culture medium in which embryo development occurs. Mature embryos are transferred to a solid medium containing selection reagent for embryo germination. The T0 plants are transferred to soil and grown in a growth room. Samples of T0 and T1 plants are analyzed by sequencing the targeted sites. Phenotype of edited variants are analyzed.

### Plant Growth

Canola plants were grown in the growth room with a 16 h photoperiod and a day/night temperature regime of 22±2° C./18±2° C. The plants were moved to the growth room with temperature regime 15±2° C./10±2° C. (day and night) when plants started to bolt.

### Microspore Culture, Embryo Selection and Plant Regeneration

Flower buds were collected with microspores at around late uninucleate development stage. The buds were immersed in 70% ethanol for about 30 seconds and then sterilized in 5% sodium hypochlorite solution (100% commercial bleach solution) and let sit for 15 to 20 minutes. The buds were then rinsed in sterile water for three times with 3 to 5 minutes each time. Microspores were isolated by maceration using a blending and filtration method. The blending and wash medium was IM-17M+10 mM K-citrate. The microspores were washed three times by centrifuge at about 1,000 revolutions per minute (rpm) for 7 minutes. Microspores were cultured in IM-17M supplemented with 15 mM CaCl₂ for 2-3 days at 31.5-33° C.

The cultured microspores were collected and loaded pieces of Nitex sieves (about 2×2 cm in size) with pore size 15 μm. The Nitex sieve and microspores complex were cultured on 3.2 cm Whatman No. 1 filter paper containing NLN-10S medium for one day at 25° C. The Nitex sieve and microspores complex were then cultured on B5 osmotic medium surface for about one hour before bombardment. Plasmid DNA with selectable marker the glyphosate acetyl transferase (GAT) gene confers resistance to glyphosate (Castle et al. (2004) Science 304:1151-1154), Cas9 and gRNAs for mutation of canola FAD2 and FAD3 gene were coated onto gold particles (0.6 μm) using CaCl2 and spermidine. 3-9 pg DNA per base pair was used in each preparation. The rupture disk used in bombardment was 650 psi. After bombardment, microspores were cultured on B5 osmotic medium for 5 hours to overnight, and then cultured for 2-3 days in NLN-13S medium with 5 μM trifluralin for chromosome doubling. The bombarded microspores were then cultured in NLN-10S with 0.1-0.2 mM glyphosate at 25° C. for putative transgenic embryo development.

Embryos developed in NLN-10S with 0.1 mM glyphosate were transferred to BGM+0.1 mM glyphosate+0.25 mg/l PCIB. The embryos on the medium were treated for two weeks in a fridge at 4° C., then cultured at 22° C. under light. The regenerated shoots were cultured on B5+GA+B for producing plantlets. 234 plantlets were transferred to soil (Table 21). Leaf tissue was sampled for sequencing the targeted sites.

TABLE 21
Microspore-derived embryo and plant production

| Experiments | Embryos | Plants |
|---|---|---|
| 16FAD01 | 293 | 93 |
| 16FAD02 | 145 | 71 |
| 16FAD03 | 348 | 57 |
| 16FAD05 | 126 | 9 |
| 16FAD06 | 219 | 4 |
| Total | 1131 | 234 |

### Target Site Sequencing Analysis in T0 and T1 Plants and Fatty Acid Profile in T1 and T2 Seeds

Three of the major fatty acids (FAs) in canola oil are the monounsaturated FA oleic acid, and the two polyunsaturated FAs linoleic acid and linolenic acid. The standard canola oil contains about 60% oleic acid (C18:1), 20% linoleic acid (C18:2) and 10% linolenic acid (C18:3). High concentrations of linolenic acid lead to oil instability and off-type flavor, while high levels of oleic acid increase oxidative stability and nutritional value of oil. The major locus for high C18:1 was proven to be the fatty acid desaturase-2 (fad2) gene and it is located on the linkage group N5. One major QTL for C18:3 is the fatty acid desaturase-3 gene of the genome C (fad3c) and it is located on N14 (Hu et al., 2006 Theor Appl Genet 113:497-507).

Canola microspores were bombarded with expression cassette DNA of selectable marker, Cas9 and gRNA. After selection in liquid medium and solid medium of the bombarded microspores and regenerated plantlets, 234 plants survived selection and were transferred to soil. The leaves of the plants were sampled to extract DNA for target site sequencing. 26 plants had indels (insertion or deletion) on the target sites of either FAD2 and/or FAD3 genes. The plant frequency with indels was 11% (26/234) (Table 22).

TABLE 22
Listing of plant variants

| | T0 | T0 | | | |
|---|---|---|---|---|---|
| Total | plants | plants | T1 seed | Plant | |
| number of | with | with T1 | FAP | variants for | T2 seeds |
| T0 plants | indels | seeds | analysis | sequencing | FAP |
| 234 | 26 | 18 | 18 | 18 | 6 |
| plants | variants | variants | variants | variants | variants |

Some plants had more than one indels. 1-nt insertion frequency was 48.9%. 1-nt deletion frequency was 13.3% (Table 23).

TABLE 23
Summary of gene edits at various target sites.

| | Target | Edited | |
|---|---|---|---|
| FAD2/FAD3 | Site (TS) | sequences | Frequency |
| Bna-FAD2-CR1 | TS1 | 4 | 8.90% |
| Bna-FAD2-CR1 | TS2 | 4 | 8.90% |
| Bna-FAD2-CR1 | TS3 | 1 | 2.20% |
| Bna-FAD2-CR1 | TS4 | 6 | 13.30% |
| Bna-FAD2-CR1 | TS5 | 1 | 2.20% |
| Bna-FAD2-CR1 | TS6 | 4 | 8.90% |
| Bna-FAD2-CR1 | TS5 | 1 | 2.20% |
| Bna-FAD2-CR1 | TS5 | 3 | 6.70% |
| Bna-FAD2-CR1 | TS6 | 1 | 2.20% |
| Bna-FAD2-CR1 | TS5 (outside cut) | 1 | 2.20% |
| BNA-FAD3-CR1 | TS1 | 6 | 13.40% |
| BNA-FAD3-CR1 | TS1 | 1 | 2.20% |
| BNA-FAD3-CR1 | TS2 | 4 | 8.90% |
| BNA-FAD3-CR1 | TS1 | 6 | 13.30% |
| BNA-FAD3-CR1 | TS1 | 2 | 4.40% |
| | 1-nt insertion | 22 | 48.90% |
| | 1-nt deletion | 6 | 13.30% |
| | 2-nt deletion | 3 | 6.70% |
| | 3-nt deletion | 4 | 8.90% |
| | Others | 10 | 22.20% |
| | | 45 | 100% |

Most variants had indels on FAD2 (69.2%). Same target site sequence (TS) but with difference in edits are shown for that TS. Other variants had indels on FAD3 (15.4%) and both FAD2 and FAD3 (15.4%) (Table 24).

TABLE 24
Listing of edited variants.

| Total | FAD2 | FAD3 | FAD2 + FAD3 |
|---|---|---|---|
| variants | variants | variants | variants |
| 26 | 18 (69.2%) | 4 (15.4%) | 4 (15.4%) |

In total 26 variants with indels, 18 plants were kept to produce T1 seeds for further sequencing analysis. Other 8 plants were haploids and were discarded without chromosome doubling treatment.

T1 seeds from 18 T0 variants were analyzed for fatty acid profile (FAP). About 70 samples from each variant were analyzed. 1141 T1 cotyledon samples were analyzed. 384 T2 seed samples were analyzed. The cotyledons of germinating T1 seeds were used for FAP analysis. The T1 seeds were germinated on wet papers for two days at ambient temperature (about 22° C.). The outer cotyledons from the germinating seeds were excised and put in gas chromatography (GC) vials. The outer cotyledons were crushed. The remaining of the germinating seeds were transferred to soil to produce plants. For analyzing FAP of T2 seeds, single seeds were added in each GC vials. Seeds were crushed. 700 μl (cotyledons) or 1000 μl of n-hexane was added in each sample vial. After about 15 minutes of incubation at ambient temperature, 100 μl of 0.5 N NaOMe solution was added with stirring a few times. After 15 minutes of standing, add 380 μl of 0.3% acetic acid to each vial. FAP was analyzed by GC. Results of FAP from T1 cotyledons showed that 6 variants had oleic acid higher than 70% (Table 25). 12 T1 plants from six variants were selected for further FAP analysis based on sequencing data, FAP data and plant growth of T1 plants.

TABLE 25
Variant phenotypes.

| | | Highest | Highest |
|---|---|---|---|
| | | oleic in | oleic in |
| Variants | T0 Indels | T1 (%) | T2 (%) |
| 16FAD1-022 | FAD2 | 67.94 | |
| 16FAD1-026 | FAD2 | 71.67 | 66.48 |
| 16FAD1-074 | FAD2 + FAD3 | 67.59 | 67.08 |
| 16FAD1-098 | FAD2 | 68.17 | |
| 16FAD1-100 | FAD2 + FAD3 | 70.5 | |
| 16FAD1-105 | FAD2 + FAD3 | 73.17 | 78.77 |
| 16FAD1-108 | FAD2 | 67.93 | |
| 16FAD1-137 | FAD2 | 63.21 | |
| 16FAD1-150 | FAD2 | 65.03 | |
| 16FAD1-160 | FAD2 | 68.88 | |
| 16FAD1-193 | FAD2 | 66.19 | |
| 16FAD1-197 | FAD2 | 68.38 | |
| 16FAD2-042 | FAD2 | 64.64 | |
| 16FAD2-066 | FAD3 | 70.42 | 63.67 |
| 16FAD2-094 | FAD2 + FAD3 | 72.04 | 65.49 |
| 16FAD2-107 | FAD2 | 71.21 | 70.58 |
| 16FAD3-030 | FAD2 | 67.9 | |
| 16FAD5-005 | FAD3 | 65.02 | |

In six variants, two variants had oleic acid concentration in T2 seeds higher than 70% (Table 26, 27). All 7 T1 plants from one variant had T2 seeds with oleic acid concentration higher than 70%.

TABLE 26
List of edited plants.

| | T1 plant | T2 seeds/ | C18:1 > | |
|---|---|---|---|---|
| Variants | # | T1 plant | 70% | Indels |
| 16FAD1-026 | 365 | 32 | 0 | FAD2 |
| 16FAD1-074 | 279 | 32 | 0 | FAD2 + FAD3 |
| 16FAD1-105 | 186 | 32 | 11 | FAD2 + FAD3 |
| | 197 | 32 | 9 | FAD2 + FAD3 |
| | 198 | 32 | 32 | FAD2 + FAD3 |
| | 213 | 32 | 32 | FAD2 + FAD3 |
| | 215 | 32 | 6 | FAD2 + FAD3 |
| | 218 | 32 | 1 | FAD2 + FAD3 |
| | 312 | 32 | 14 | FAD2 + FAD3 |
| 16FAD2-066 | 279 | 32 | 0 | FAD3 |
| 16FAD2-094 | 208 | 32 | 0 | FAD2 + FAD3 |
| 16FAD2-107 | 307 | 32 | 3 | FAD2 |

TABLE 27
List of edited plants.

| | T2 seeds/ | | | |
|---|---|---|---|---|
| T1 plants | T1 plant | C18:1 (%) | C18:2 (%) | C18:3 (%) |
| 16FAD1- | 32 | 75.1 | 11.1 | 5.0 |
| 105_198 | | | | |
| 16FAD1- | 32 | 76.4 | 10.5 | 4.6 |
| 105_213 | | | | |

### Protocols

### Production of Pre-Incubated Microspores

Microspores are cultured as is known to those skilled in the art, for example see Fukuoka et al. (1996) Plant Physiol 111:39-47; Keller et al. (1987) Proc. 7th Int. Rapeseed Congr. (Plant Breeding and Acclimatization Institute, Poznan, Poland) pp. 152-157, Swanson et al. (1987) Plant Cell Reports 6: 94-97 and Baillie et al. (1992) Plant Cell Reports 11: 234-237. A detailed procedure is provided as follows:

Collect about 400 buds at uni-nucleus microspore stage from a Brassica variety that is responsive to microspore culture and regeneration, for example 46A65, Westar or Topas. Sterilize the buds in 5% sodium hypochlorite solution (100% commercial bleach solution) and let sit for 15-20 minutes. Place the buds in sterile water for 5 minutes to rinse off bleach. Repeat this step two more times. Empty buds into blender cups and blend for 8 seconds at low speed in 20-25 ml B5-W. Filter contents through two nested 44 μm Nitex filters into 50 ml centrifuge tubes. Wash filters with 20-25 ml B5-W, cap tubes and centrifuge at about 1,000 revolutions per minute (rpm) for 6 minutes. Decant B5-W, add 45 ml B5-W, centrifuge, decant, add 45 ml B5-W, centrifuge and repeat for a total of 4 washes. Before plating microspores, adjust microspore density to 100,000 microspores per ml with NLN-17S using a heamocytometer. Plate microspore suspension in 9-cm plates at 6 ml/plate. Culture the plates (around 40) for 2-3 days at 31.5° C., followed by NLN-10S. Culture the plates at 25° C. for an additional 3-4 days.

### Particle Bombardment

Methods of particle bombardment are used to deliver the genome editing system.

### Make Gold Particle Aliquots

Use 250 mg gold particles with diameter of 0.6 micron. Add 1000 μl EtOH and sonicate 5-8 seconds. Divide gold suspension to two tubes (500 μl each) (Fisher 05-541-27). Centrifuge for one minute, 13,000 rpm and pipette off EtOH. Wash with 1 ml sterile distilled water three times. Wash gold pellets with 1000 μl EtOH and Pipette off EtOH. Add 800 μl EtOH to each tube and suspend gold particles. Weigh 16 tubes (Fisher 05-541-27). Aliquot 100 μl gold suspension to each tube. Centrifuge 30 seconds and pipette off EtOH. Dry for one hour and weigh tube/gold to calculate gold weight in each tube. Add sterile water to each tube to make gold concentration to 3 mg/50 μl. Suspend gold particles with pipette sucking-releasing and sonication (3 seconds). Aliquot gold suspension to new 1.5 ml tubes at 50 ul/tube (3 mg/tube). Store in −20 C. freezer.

### Coat DNA on Gold Particles

Gold particle amount per shot is 100 μg/shot (3 mg/30 shots). Add 3 μg DNA/bp/prep plasmid DNA, 50 μl CaCl2 (2.5 M, aliquoted into small volumes), 20 μl spermidine (0.1 M, base-free, aliquoted into small volumes) to a tube of gold particle aliquot. Pipette 30-50 times after each addition. Shake for 3 minutes on vortex shaker. Centrifuge for 10 seconds at 10,000 rpm and discard supernatant. Gently add 200 μl 100% EtOH and set in ice for 10 minutes and discard supernatant. Wash gently with 200 μl 100% EtOH twice and discard washes. Add 150 μl 100% EtOH. Use 10 μl pipette tip to suspend the gold pellet. Fully suspend gold particles. Sonicate for 3 one-second dips to break small pellets. Sterilize macro-carrier discs in 70% EtOH for minimum 10 minutes. Transfer to 100% EtOH then dry in laminar flow cabinet. Place 5 μl DNA-coated gold particle suspension on the centre of each sterilized macro-carrier disc and dry for at least half an hour.

### Biolistic Gun Operation

The microcarrier launch assembly parts, the macrocarrier holders, the rupture retaining cap, macroccarrier, petri dish holder and the stopping screens can be sterilized either by soaking (or spraying) in 70% EtOH for 15 minutes and drying in the laminar flow cabinet, or by autoclaving. The rupture disks should be sterilized in 50% iso-propanol for 10-30 seconds. Sterilize the chamber by spraying 70% EtOH. Build up helium pressure higher than rupture disk. Load the rupture disk retaining cap and microcarrier launch assembly and assemble in chamber. Place the microcarrier launch assembly in the first slot from the top. Position the sample on the petri dish holder at the third slot. Close the chamber. Set the vacuum switch on the gun to VAC position. Once the vacuum level is reached to or beyond 27 inch Hg, place the vacuum switch in the HOLD position. Press and hold FIRE switch until burst. Release the fire switch immediately and switch the VACUUM to the VENT position. After the vacuum is released, take out sample and microcarrier launch assembly. Repeat until all samples are bombarded.

### Bombardment and Selection of Pre-Incubated Microspores

Culture microspores for 6-8 days using NLN-17S/10S protocol. Collect microspores with Nitex nylon sieve with 25 μm in pore size. Transfer microspores to a piece of Nitex nylon sieve and blot extra medium with filter papers. Transfer microspores and the Nitex to osmotic medium (B5+17% sucrose+1 g/l MES+0.8% gelrite, pH: 6.0) and treat at least one hour. Bombard samples with rupture disk 650 psi or 900 psi. Leave samples in the osmotic medium for at least 4 hours. Transfer microspores and Nitex to NLN-13S. A chromosome doubling agent can be added. Medium contains no selection agent (nptll selection) or 0.1-0.2 mM glyphosate (GAT selection). Each plate (9 cm) contains 5 ml medium and one piece of microspores/Nitex. Culture the bombarded microspores for 7 days at 25 C. in dark. Replace medium with 10 ml of NLN-6.5S with final concentration of NAA0.5BAP0.05+G418 (10 mg/l) if using the nptll gene as selectable marker or add 5 ml of NLN-OS with Glyphosate 0.1-0.2 mM if using the gat gene as selectable marker. Culture under light for 2 weeks.

### Plant Regeneration

Culture green embryos or tissue in MMW+IAA2+TDZ0.5+STS6 with 25 mg/l kanamycin or 0.1 mM glyphosate for 4 weeks (STSG is silver thiosulfate at concentration of 6 μM). Isolate regenerated buds and culture in MMW+BAP0.2 or B5+GA with 50 mg/l kanamycin or 0.1 mM glyphosate. Excise shoots and transfer to rooting medium ½ MMW+1%sucrose+IBA2 with 25 mg/l kanamycin or B5+GA with 0.1 mM glyphosate.

### Transfer Resistant Plants to Soil and PCR Assay

Transfer resistant plants to soil in 36-cell flats after root is well developed. To maintain humidity, place lid on top for one week or until shoots have established themselves in soil. Assay for transgenics by PCR analysis using a PCR kit from Sigma (REDExtract-N-Amp Plant PCR kit). Transgenic plants must be labeled as “transgenic” or “GMO”.

### GUS Assay

GUS analysis is known to those skilled in the art. The protocol can be found in numerous references, for example Wu H, McCormac A C, Elliott M C, Chen D F (1998) Agrobacterium-mediated s table transformation of cell suspension cultures of barley (Hordeum vulgare). Plant Cell Tissue and Organ Culture 54:161-171.

### PCR Analysis of the GAT and NPTII Genes

PCR analysis is known to those skilled in the art. The protocol can be found in numerous references. For example, PCR analysis of the nptII gene was done according to Broothaerts W, Wiersma P A, Lane W D (2001) Multiplex PCR combining transgene and S-allele control primers to simultaneously confirm cultivar identity and transformation in apple. Plant Cell Rep 20:349-353.

Plant DNA was extracted following Sigma™ Technical Bulletin Code MB-850 and using REDExtract-N-Amp™ Plant PCR kit. The temperature cycle was 95° C., 2 min; (94° C., 30 s; 64° C., 30 s; 72° C., 30 s) for 35 cycles; 75° C., 5′ for the GAT gene amplification, or 95° C., 2 min; (95° C., 15 s; 60° C., 30 s; 72° C., 30 s) for 35 cycles; 75° C., 5′ for the nptll gene. Product Size: GAT4604 and 4618: 317 bp; GAT4621: 255 bp; NPTII: 700 bp.

### MEDIA RECIPES

### MMW

MS salts and organics (Murashige and Skoog 1962 Physiol. Plant. 15:473-479)
Sucrose (3%)
MES (2 g/l)
Sigma agar #1296 (0.6%) pH 5.8

### MMW+IAA+TDZ+AgNO3+Gly

MMW
IAA (2 mg/l)
TDZ (0.5 mg/l)
silver nitrate (5 mg/l)
glyphosate (0.1 mM)

### MMW+BAP+Kan 25-50

MMW
BAP (4 mg/l)
Kanamycin (25-50 mg/l)

### MMW+kan 50-100

MMW
Kanamycin (50-100 mg/l)

### B5

B5 vitamins and minerals (Gamborg et al. (1968) Exp. Cell Res. 50:151-158)
Sucrose (2%)
Sigma agar (0.6%)
pH 5.8

### B5+GA+Gly

B5
GA3 (0.1 mg/l)
Glyphosate (0.1 mM)

### B5-W

B5 (no agar)
Sucrose (130 g/l)

### NLN

Components are as Lichter (1982) Z Pflanzenphysiol 105:427-434 without potato broth and plant growth regulators. Medium pH is 6.0.

### NLN-17S, NLN-10S, NLN-6.5S

NLN contains 17%, 10% or 6.5% sucrose

### Osmotic Medium for Pre-Incubated Microspores

B5
Sucrose 170 g/l
MES (1 g/l)
Phytagel (8-16 g/l)
pH: 6.0
   Rooting Medium (½ MMW+1% sucrose+2 IBA)
Half strength MMW
Sucrose (10 g/l)
IBA (2 mg/lI)

TABLE 28
IM-17M

| Components | mg/L |
|---|---|
| KNO3 | 125 |
| MgSO4•7H2O• | 125 |
| Ca(NO3)2•4H2O | 500 |
| KH2PO4 | 125 |
| MnSO4•H2O | 16.9 |
| H3BO3 | 6.2 |
| ZnSO4•7H2O | 8.6 |
| Na2MoO4•2H2O | 0.25 |
| CuSO4•5H2O | 0.025 |
| CoCl2•6H2O | 0.025 |
| Myo-insitol | 100 |
| Nicotinic acid | 5 |
| Pyridoxine HCL | 0.5 |
| Thiamine HCL | 0.5 |
| Biotin | 0.05 |
| Glycine | 2 |
| Folic acid | 0.5 |
| Glutathione | 30 |
| Maltose monohydrate | 170,000 |
| pH | 6.5 |

TABLE 29
IM-17M + 10 mM K-citrate

| Components | mg/L |
|---|---|
| KNO3 | 125 |
| MgSO4•7H2O• | 125 |
| Ca(NO3)2•4H2O | 500 |
| KH2PO4 | 125 |
| MnSO4•H2O | 16.9 |
| H3BO3 | 6.2 |
| ZnSO4•7H2O | 8.6 |
| Na2MoO4•2H2O | 0.25 |
| CuSO4•5H2O | 0.025 |
| CoCl2•6H2O | 0.025 |
| Myo-insitol | 100 |
| Nicotinic acid | 5 |
| Pyridoxine HCL | 0.5 |
| Thiamine HCL | 0.5 |
| Biotin | 0.05 |
| Glycine | 2 |
| Folic acid | 0.5 |
| Glutathione | 30 |
| Potassium citrate tribasic monohydrate | 3,240 |
| Maltose monohydrate | 170,000 |
| pH | 7.0 |

TABLE 30
NLN-10S

| Components | mg/l |
|---|---|
| Boric Acid | 10 |
| Calcium Nitrate | 347 |
| Cobalt Chloride•6H2O | 0.025 |
| Cupric Sulfate•5H2O | 0.025 |
| D-Biotin | 0.05 |
| Ferric Sodium EDTA | 36.7 |
| Folic Acid | 0.5 |
| Glutathione | 30 |
| Glycine (Free Base) | 2 |
| L-Glutamine | 800 |
| L-Serine | 100 |
| Magnesium Sulfate, Anhydrous | 61 |
| Manganese Sulfate•H2O | 18.95 |
| Molybdic Acid (Sodium Salt)•2H2O | 0.25 |
| myo-Inositol | 100 |
| Nicotinic Acid (Free Acid) | 5 |
| Potassium Nitrate | 125 |
| Potassium Phosphate, Monobasic | 125 |
| Pyridoxine•HCI | 0.5 |
| Thiamine•HCI | 0.5 |
| Zinc Sulfate•7H2O | 10 |
| Sucrose | 100,000 |
| pH | 6.0 |

TABLE 31
NLN-13S

| Components | mg/l |
|---|---|
| Boric Acid | 10 |
| Calcium Nitrate | 347 |
| Cobalt Chloride•6H2O | 0.025 |
| Cupric Sulfate•5H2O | 0.025 |
| D-Biotin | 0.05 |
| Ferric Sodium EDTA | 36.7 |
| Folic Acid | 0.5 |
| Glutathione | 30 |
| Glycine (Free Base) | 2 |
| L-Glutamine | 800 |
| L-Serine | 100 |
| Magnesium Sulfate, Anhydrous | 61 |
| Manganese Sulfate•H2O | 18.95 |
| Molybdic Acid (Sodium Salt)•2H2O | 0.25 |
| myo-Inositol | 100 |
| Nicotinic Acid (Free Acid) | 5 |
| Potassium Nitrate | 125 |
| Potassium Phosphate, Monobasic | 125 |
| Pyridoxine•HCI | 0.5 |
| Thiamine•HCI | 0.5 |
| Zinc Sulfate•7H2O | 10 |
| Sucrose | 130,000 |
| pH | 6.0 |

TABLE 32
B5 osmotic medium

| Components | mg/l |
|---|---|
| Ammonium Sulfate | 134 |
| Boric Acid | 3 |
| Calcium Chloride, Anhydrous | 113.24 |
| Cobalt Chloride•6H2O | 0.025 |
| Cupric Sulfate•5H2O | 0.025 |
| Ferrous Sulfate•7H2O | 27.8 |
| Na2 EDTA•2H2O | 37.3 |
| Magnesium Sulfate, Anhydrous | 122.09 |
| Manganese Sulfate•H2O | 10 |
| Molybdic Acid (Sodium Salt)•2H2O | 0.25 |
| Potassium Iodide | 0.75 |
| Potassium Nitrate | 2500 |
| Sodium Phosphate Monobasic | 150 |
| Zinc Sulfate•7H2O | 2 |
| myo-Inositol | 100 |
| Nicotinic Acid (Free Acid) | 1 |
| Pyridoxine•HCI | 1 |
| Thiamine•HCI | 10 |
| Sucrose | 150,000 |
| 2-(N-Morpholino)ethanesulfonic acid hydrate | 500 |
| 2-(N-Morpholino)ethanesulfonic acid potassium salt | 500 |
| pH | 6.0 |
| Phytagel | 14,000 |

TABLE 33
BGM + 0.25 mg/l PCIB + 0.1 mM Gly

| Components | mg/l |
|---|---|
| Ammonium Sulfate | 67 |
| Boric Acid | 3 |
| Calcium Chloride, Anhydrous | 56.62 |
| Cobalt Chloride•6H2O | 0.025 |
| Cupric Sulfate•5H2O | 0.025 |
| Ferrous Sulfate•7H2O | 13.9 |
| Na2 EDTA•2H2O | 18.65 |
| Magnesium Sulfate, Anhydrous | 61.045 |
| Manganese Sulfate•H2O | 10 |
| Molybdic Acid (Sodium Salt)•2H2O | 0.25 |
| Potassium Iodide | 0.75 |
| Potassium Nitrate | 1250 |
| Sodium Phosphate Monobasic | 150 |
| Zinc Sulfate•7H2O | 2 |
| myo-Inositol | 12.5 |
| Nicotinic Acid (Free Acid) | 0.125 |
| Pyridoxine•HCI | 0.125 |
| Thiamine•HCI | 1.25 |
| PCIB (2-(p-Chlorophenoxy)-2-methylpropionic acid) | 0.25 |
| Glyphosate | 16.9 |
| Sucrose | 5,000 |
| Agar | 6,000 |
| pH | 5.8 |

TABLE 34
B5 + GA + B

| Components | mg/l |
|---|---|
| Ammonium Sulfate | 134 |
| Boric Acid | 3 |
| Calcium Chloride, Anhydrous | 113.24 |
| Cobalt Chloride•6H2O | 0.025 |
| Cupric Sulfate•5H2O | 0.025 |
| Ferrous Sulfate•7H2O | 27.8 |
| Na2 EDTA•2H2O | 37.3 |
| Magnesium Sulfate, Anhydrous | 122.09 |
| Manganese Sulfate•H2O | 10 |
| Molybdic Acid (Sodium Salt)•2H2O | 0.25 |
| Potassium Iodide | 0.75 |
| Potassium Nitrate | 2500 |
| Sodium Phosphate Monobasic | 150 |
| Zinc Sulfate•7H2O | 2 |
| myo-Inositol | 100 |
| Nicotinic Acid (Free Acid) | 1 |
| Pyridoxine•HCI | 1 |
| Thiamine•HCI | 10 |
| Sucrose | 20,000 |
| Gibberellic acid | 0.1 |
| Benlate | 5 |
| Agar | 6 |
| pH | 5.8 |

*Trade-mark

### EXAMPLE 2

### Optimizing Transformation Procedures for Gene Editing

DNA constructs with promoter AT-UBIQ10 and glyphosate resistant gene GAT4621 were used in the following experiments.
   1. Effect of Culture Medium and Period Before Bombardment on Transformation Efficiency
The highest microspore embryogenesis was produced when microspores were cultured in IM-17M for 2 to 3 days at 31.5° C. In developing canola transformation procedure, microspores were cultured for two days or three days in IM-17M and then cultured in NLN-10S for one day or not before bombardment. It was found that one day of culture in NLN-10S had effect on transformation efficiency (Table 35).

TABLE 35
Microspore culture time and media on
resistant embryo and plant production

| Culture in | Culture in | | Embryos/ | Plant/ |
|---|---|---|---|---|
| IM-17M | NLN-10S | # shots | shot | shot |
| 2 days | 0 day | 115 | 1.7 | 0.2 |
| 2 days | 1 day | 156 | 14.3 | 9.9 |
| 3 days | 1 day | 120 | 5.0 | 2.3 |

Three days of microspore culture at 31.5° C. had lower resistant embryo production than two days of culture. Reducing temperature on the 3^{rd} day culture from 31.5° C. to 25° C. increased resistant embryo production (Table 36).

TABLE 36
Microspore culture temperature on resistant embryo production

| Microspore culture | | | Embryos/ |
|---|---|---|---|
| temperature | # shots | # embryos | shot |
| 2 d, 31.5° C. | 47 | 727 | 16.1 |
| 2 d, 31.5° C.; 1 d, 25° C. | 49 | 1095 | 23.0 |

2. Effect of CaC12 in IM-17M before bombardment and NLN-13S with 5 μM trifluralin on transformation efficiency
15 mM calcium chloride was supplemented in IM-17M in microspore culture before bombardment. It was found that resistant embryo number was increased in the medium supplemented with 15 mM CaCl2 (Table 37).

TABLE 37
Calcium chloride in IM-17M medium on transformation efficiency

| Microspore culture | | | Embryos/ |
|---|---|---|---|
| medium | # shots | # embryos | shot |
| IM-17M | 24 | 375 | 15.6 |
| IM-17M + 15 mM CaCl2 | 24 | 574 | 23.9 |

It was further studied that calcium chloride was supplemented in NLN-13S with 5 μM trifluralin after bombardment on transformation efficiency. Result showed that supplement of calcium chloride in NLN-13S+trifluralin medium produced more resistant embryos (Table 38).

TABLE 38
Calcium chloride in NLN-13S +
trifluralin on transformation efficiency

| | CaCl₂ in | | | |
|---|---|---|---|---|
| CaCl₂ in | NLN-13S + | | | Embryos/ |
| IM-17M | trifluralin | # embryos | # shots | shot |
| 15 mM | 0 mM | 139 | 22 | 6 |
| 0 mM | 10 mM | 169 | 15 | 11 |
| 0 mM | 20 mM | 300 | 19 | 16 |

3. DNA concentration on resistant embryo production and editing frequency
Expression cassette DNA concentration 3 pg/bp/preparation was usually used in canola biolistic transformation. Increasing DNA concentration by 3-fold did not reduce resistant embryo production and increased editing frequency (Table 39).

TABLE 39
DNA concentration on resistant embryo
production and gene editing frequency

| DNA concentration of | | | | |
|---|---|---|---|---|
| 3 constructs | Embryos/ | | Edited | Editing |
| GAT4621:FAD2:FAD3 | shot | Plants | plants | frequency |
| 3:9:9 pg/bp/preparation | 11.6 | 88 | 6 | 6.8% |
| 9:27:27 pg/bp/preparation | 12.2 | 75 | 11 | 14.7% |
| 27:81:81 pg/bp/preparation | 9.5 | 58 | 9 | 15.5% |

4. Increase embryo germination frequency
One of technical challenges in canola transformation was that embryo germination frequency was only around 20%. Antiauxin 2-(p-Chlorophenoxy)-2-methylpropionic acid (PCIB) was included in embryo germination medium BGM for inhibiting callusing. Embryo germination frequency was doubled when 0.0625-1 mg/l PCIB was added in BGM with 0.1 mM glyphosate

(Table 42).

TABLE 40
Effect of PCIB in embryo germination
medium on germination frequency

| PCIB concentration | # | # | Germination |
|---|---|---|---|
| (mg/l) | embryos | plantlets | frequency |
| 0 | 72 | 17 | 24% |
| 0.0625 | 63 | 28 | 44% |
| 0.125 | 72 | 31 | 43% |
| 0.25 | 72 | 36 | 50% |
| 0.5 mg/l | 63 | 28 | 44% |
| 1 mg/l | 81 | 36 | 44% |

To further increase embryo germination frequency, abscisic acid (ABA) at 25 μM was added in microspore culture medium NLN-10S with 0.1 -0.2 mM glyphosate 14 days after bombardment. The resistant embryos were partially desiccated in a Petri dish for one week at 25° C. The Petri dish contained a piece of Bounty paper towel with water for maintaining moisture. After the treatment, embryo germination frequency was significantly increased (Table 43).

TABLE 41
Effect of ABA application and embryo partial
desiccation on embryo germination frequency

| | Total | Total | |
|---|---|---|---|
| Embryo treatment | embryos | shoots | Germination |
| No ABA, No partial | 60 | 7 | 11.7% |
| desiccation | | | |
| ABA, partial | 479 | 338 | 76.6% |
| desiccation | | | |

All publications and patent applications mentioned in the specification are indicative of the level of those skilled in the art to which this disclosure pertains and are incorporated by reference.

### EXAMPLE 3

### Direct Delivery of gRNA and Cas Endonuclease as a Guide RNA/Cas Endonuclease Ribonucleotide-Protein Complex (RGEN or RNP) into Brassica Microspores

This example demonstrates that direct delivery of a Cas endonuclease (e.g., Cas9) in the form of protein and gRNA in the form of in vitro transcribed or chemically synthesized RNA molecules, into Brassica microspore cells can generate site-specific targeted editing of genomes. Exemplary materials and methods for delivering ribonucleoprotein (RNP) complex into plant cells can be found in US20180327785A1 (see Examples 10-13), which is incorporated by reference.

To generate gRNA in the form of RNA molecules, a suitable in vitro transcription reaction can be carried out with a commercially available one according to the manufacturer's recommendations, and products are purified using e.g., NucAway Spin Columns (Invitrogen; Life Technologies Inc) followed by ethanol precipitation. To generate a guide RNA/Cas9 endonuclease protein complex (RGEN or RNP) (also referred to as a guide RNA/Cas9 endonuclease ribonucleotide-protein), for example, 7 μg of Cas9 (Streptococcus pyogenes Cas9) protein and 3 μg of gRNA molecules (1:2 molar ratio) are mixed in 1×Cas9 buffer (NEB) in a total volume of 20 μl and incubated at room temperature for 15 minutes. Together with the RGEN, plasmids containing a promoter regulated selectable and visible markers (e.g., MoPAT-DsRed fusion), are mixed with a particle delivery matrix comprising commercially available gold particles (0.6 μm, Bio-Rad) and a water soluble cationic lipid TransIT-2020 (Mirus, USA). The particle delivery matrix comprising the guide RNA/Cas endonuclease ribonucleotide-protein complexes are delivered into microspore cells using particle mediated delivery (as described in the preceding sections) and may include some modifications. Appropriate amount e.g., 10 μl is spotted onto the center of each macrocarrier and allowed to dry before bombardment.

Untreated Brassica microspores and microspores bombarded with the Cas9 protein only can serve as negative controls while embryos bombarded with DNA vectors expressing Cas9 and gRNA are used as positive controls. Similar frequencies are expected for Cas9-gRNA components delivered as DNA vectors and as guide RNA/Cas endonuclease ribonucleotide-protein complexes, based on the teaching and guidance provided in this disclosure.

### EXAMPLE 4

### Direct Delivery of Cas9 in the Form on mRNA and gRNA into Microspore Cells of Brassica

This example demonstrates that direct delivery of Cas9, in the form of mRNA molecules and gRNA in the form of in vitro transcribed or chemically synthesized RNA molecules, into Brassica cells generates edits at the corresponding targeted sites. Cas9 can be delivered as mRNA molecules. Commercially available Cas9 m RNA (TriLink Biotechnologies) can be used in the experiment. Brassica microspore cells are co-bombarded with Cas9 mRNA (e.g., 200 ng), gRNA in the form of in vitro synthesized RNA molecules (100 ng). Commercially available Cas9 mRNA (TriLink Biotechnologies) and RNA molecules, in vitro synthesized as described above, are used in the experiment. This demonstrates that delivery of both Cas9 and gRNA, in the form of RNA molecules, can generate targeted mutations and, along with Cas9-gRNA delivery as the RGEN complex, is an alternative to DNA delivery for targeted mutagenesis and gene editing in Brassica.

We claim:

1. A method of editing genome of a Brassica plant cell, the method comprising:
a. culturing isolated microspore from a Brassica plant to produce an isolated pre-incubated microspore;
b. culturing the isolated pre-incubated microspore under conditions of plasmolysis;
c. introducing, under conditions of plasmolysis through bombardment a microparticle comprising a CRISPR-Cas genome editing system into the isolated pre-incubated microspore;
d. culturing under conditions of plasmolysis the pre-incubated microspore comprising a genetic modification introduced by the genome editing system to generate a transformed microspore;
e. culturing the transformed pre-incubated microspore in or on a liquid selection medium to produce an embryo or a tissue; and
f. regenerating a Brassica plant from the embryo or the tissue, wherein the Brassica plant comprises the genetic modification introduced by the genome editing system.

2. The method of claim 1, wherein the genome editing system comprises CRISPR/Cas endonuclease selected from the group consisting of Cas9, Cpf1, or Csm1.

3. The method of claim 2, wherein the genome editing system comprises a guide RNA, a Cas endonuclease, and combination thereof.

4. The method of claim 2, wherein the genome editing system comprises a DNA construct that encodes Cas endonuclease, a guide RNA or a combination thereof.

5. The method of claim 2, wherein the DNA construct comprises both a guide RNA expression cassette and a Cas endonuclease expression cassette.

6. The method of claim 1, wherein the gene-editing system comprises a ribonucleoprotein complexed with a guide RNA on a microparticle.

7. The method of claim 1, wherein the genome editing system comprises guide RNA and a donor DNA template.

8. The method of claim 1, wherein the embryo is treated with a chromosome doubling agent.

9. A method of increasing genome editing frequency of Brassica, the method comprising:
a. culturing isolated microspore obtained from a Brassica plant to produce an isolated pre-incubated microspore that has been conditioned to be embryogenic;
b. introducing, under conditions of plasmolysis, a microparticle carrier comprising a targeted genome editing system into the isolated pre-incubated microspore through bombardment, wherein the microspore does not burst or otherwise damaged upon receiving the microparticle;
c. generating an embryo from the bombarded microspore under suitable culturing conditions; and
d. generating a Brassica plant from the embryo comprising a genetic modification introduced by the genome editing system.

10. The method of claim 9, wherein the genome editing system is a CRISPR/Cas gene editing system.

11. The method of claim 10, wherein the DNA construct comprises a guide RNA expression cassette or a Cas endonuclease expression cassette.

12. The method of claim 10, wherein the genome editing system comprises a ribonucleoprotein (RNP) complex that is capable of site-directed editing of endogenous genomic DNA.

13. The method of claim 11, wherein the genetic modification is introduction of a heterologous DNA through homologous recombination or homology mediated repair.

14. The method of claim 10, wherein the gene-editing system targets Brassica FAD2, FAD3 or a combination thereof.

15. The method of claim 13, wherein the expression cassette further comprises a gene encoding a selectable marker.

16. The method of claim 15, wherein the selectable marker is glyphosate acetyltransferase.

17. An embryogenic Brassica microspore cell comprising a microparticle, wherein the microparticle delivers a genome editing complex comprising a CRISPR-Cas endonuclease, a guide RNA, and optionally a donor template, wherein the Cas endonuclease and guide RNA form a complex with a target genomic DNA in the microspore cell, wherein the microspore cell is embryogenic and capable of developing into an embryo.

18. The microspore cell of claim 17, wherein the genome comprises a targeted edit introduced by the Cas endonuclease—gRNA complex.

19. The microspore cell of claim 17, wherein the CRISPR-Cas endonuclease is one of Cas9, Cpf1, or Csm1.

20. The microspore cell of claim 17, wherein the microspore is present in an osmolytic culture medium.

21. The microspore cell of claim 17, wherein the microspore is obtained from a donor Brassica plant that is Canola.

22. The microspore cell of claim 17, wherein the microspore is tolerant to a selectable marker.

23. The microspore cell of claim 17, wherein the microspore comprises a mutation in Brassica FAD2, FAD3 or a combination thereof.
